(19) United States
(12) Patent Application Publication
(10) Pub. No.: US 2019/0372021 A1
(43) Pub. Date: Dec. 5, 2019

(54) HETEROCYCLIC COMPOUND AND ORGANIC PHOTOELECTRIC DEVICE COMPRISING SAME

(71) Applicant: LG CHEM, LTD., Seoul (KR)

(72) Inventors: JIYOUNG LEE, Daejeon (KR); SEUNG JUN YOO, Daejeon (KR); JUNGHYUN PARK, Daejeon (KR); YOUNSHIN KIM, Daejeon (KR); SANG AH KIM, Daejeon (KR)

(21) Appl. No.: 16/484,865

(22) PCT Filed: Oct. 18, 2018
(86) PCT No.: PCT/KR2018/012316

§ 371 Date: Aug. 9, 2019

(30) Foreign Application Priority Data
Nov. 15, 2017 (KR) 10-2017-0152386

(51) Int. Cl. H01L 51/00; C07D 417/04; C07D 417/14; C07D 421/14

(52) U.S. Cl. CPC H01L 51/0072; C07D 417/04; H01L 51/0071; C07D 417/14; H01L 51/4253; H01L 51/0067; C07D 421/14; H01L 51/0052; H01L 51/0068

(57) ABSTRACT

The present specification relates to a heterocyclic compound of Formula 1 and an organic photoelectric device including the same.

### CROSS REFERENCE TO RELATED APPLICATIONS

This application is a 35 U.S.C. § 371 national stage application of PCT International Application No. PCT/KR2018/012316, filed Oct. 18, 2018, which claims priority from Korean Patent Application No. 10-2017-0152386, filed Nov. 15, 2017, the contents of which are incorporated herein in their entireties by reference. The above-referenced PCT International Application was published in the Korean language as International Publication No. WO 2019/098542 on May 23, 2019.

### TECHNICAL FIELD

The present specification relates to a heterocyclic compound and an organic photoelectric device including the same.

### BACKGROUND ART

An organic photoelectric device is a device that converts light into electric signals by using the photoelectric effect, includes a photodiode, a phototransistor, and the like, and may be applied to an image sensor, and the like. In an image sensor including a photodiode, the resolution is increasing day by day, and accordingly, the pixel size is decreasing. Currently, in the case of a silicon photodiode mainly used, as the size of pixel is decreased, the absorption area is reduced, so that the reduction in sensitivity may occur. Accordingly, organic materials capable of replacing silicon have been studied.

Since organic materials have a high extinction coefficient and may selectively absorb light in a specific wavelength region according to the molecular structure, the organic materials may replace a photodiode and a color filter, and thus, are very advantageous in improving sensitivity and high integration.

### DETAILED DESCRIPTION OF THE INVENTION

### Technical Problem

The present specification provides a heterocyclic compound and an organic photoelectric device including the same.

### Technical Solution

An exemplary embodiment of the present specification provides a heterocyclic compound represented by the following Formula 1.

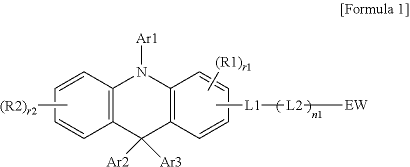

In Formula 1,

L1 and L2 are the same as or different from each other, and are each independently a substituted or unsubstituted divalent heteroaryl group,

Ar1 to Ar3 are the same as or different from each other, and are each independently a substituted or unsubstituted alkyl group; a substituted or unsubstituted aryl group; or a substituted or unsubstituted heteroaryl group,

EW has a structure that serves as an electron acceptor,

R1 to R2 are the same as or different from each other, and are each independently hydrogen; deuterium; a halogen group; a nitrile group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylamine group; or a substituted or unsubstituted aryl group,

n1 is 0 or 1,

r1 is an integer from 1 to 3,

r2 is an integer from 1 to 4,

when r1 is 2 or more, two or more R1's are the same as or different from each other, and

when r2 is 2 or more, two or more R2's are the same as or different from each other.

Further, an exemplary embodiment of the present specification provides an organic photoelectric device including: a first electrode; a second electrode disposed to face the first electrode; and an organic material layer having one or more layers disposed between the first electrode and the second electrode, in which one or more layers of the organic material layer include the above-described heterocyclic compound.

### Advantageous Effects

A heterocyclic compound according to an exemplary embodiment of the present specification serves as an electron donor, so that the dipole moment between molecules is increased to reduce the band gap, and light with a long wavelength can be absorbed by increasing the interaction between molecules. Accordingly, an organic photoelectric device including the same has excellent photoelectric conversion efficiency.

### BRIEF DESCRIPTION OF DRAWINGS

FIG. 1 is a cross-sectional view illustrating the organic photoelectric device according to an exemplary embodiment of the present specification.

FIG. 2 is an FT-NMR graph of Compound 1 of the present specification.

FIG. 3 is a view illustrating a UV-vis absorption spectrum in a solution state of Compound 1 of the present specification.

FIG. 4 is an FT-NMR graph of Compound 2-C of the present specification.

FIG. 5 is an FT-NMR graph of Compound 2 of the present specification.

FIG. 6 is a view illustrating a UV-vis absorption spectrum in a solution state of Compound 2 of the present specification.

FIG. 7 is a current density graph of the organic photoelectric device manufactured in Example 1-1 of the present specification over voltage at dark current.

FIG. 8 is a current density graph of the organic photoelectric device manufactured in Example 1-1 of the present specification over voltage at photoelectric current.

FIG. 9 is a current density graph of the organic photoelectric device manufactured in Example 1-2 of the present specification over voltage at dark current.

FIG. 10 is a current density graph of the organic photoelectric device manufactured in Example 1-2 of the present specification over voltage at photoelectric current.

FIG. 11 is an FT-NMR graph of Compound 3 of the present specification.

FIG. 12 is an FT-NMR graph of Compound 6 of the present specification.

FIG. 13 is an FT-NMR graph of Compound 12 of the present specification.

FIG. 14 is an FT-NMR graph of Compound 13 of the present specification.

FIG. 15 is data obtained by measuring the UV-vis absorption spectra in the solution and film states of Compound 3 of the present specification.

FIG. 16 is data obtained by measuring the UV-vis absorption spectra in the solution and film states of Compound 12 of the present specification.

FIG. 17 is data obtained by measuring the UV-vis absorption spectra in the solution and film states of Compound 14 of the present specification.

FIG. 18 is a current density graph of the organic photoelectric devices manufactured in Examples 6-1 and 6-2 of the present specification over voltage at photoelectric current and dark current.

FIG. 19 is a graph illustrating the external quantum efficiencies of the organic photoelectric devices manufactured in Examples 6-1 and 6-2 of the present specification over wavelength and voltage.

FIG. 20 is a top view of the organic photoelectric devices manufactured in Examples 1-1, 1-2, 2-1, 2-2, 3-1, 3-2, 4-1, 4-2, 5-1, 5-2, 6-1, and 6-2 of the present specification.

### EXPLANATION OF REFERENCE NUMERALS AND SYMBOLS

10: First electrode
20: Second electrode
30: Photoactive layer
100: Organic photoelectric device
1: Anode (Cathode)
2: Organic material layer
3: Cathode (Anode)

### BEST MODE

Hereinafter, the present specification will be described in detail.

The present specification provides the heterocyclic compound represented by Formula 1.

Since the heterocyclic compound represented by Formula 1 according to an exemplary embodiment of the present specification effectively achieves intermolecular stacking by including acridine which serves as an electron donor to adjust the molecular planarity, the wavelength width of light absorbed is large. Further, the heterocyclic compound represented by Formula 1 can absorb light at a long wavelength by inserting a thiophene group which serves as an electron donor and/or a benzothiadiazole group which serves as an electron acceptor into a linking group to increase intermolecular interaction.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

In the present specification, the electron donor also refers to an electron donating body, and generally means having a negative charge or an unshared electron pair, and donating electrons to a portion in which a positive charge or an electron pair lacks. Additionally, the electron donor in the present specification includes those capable of transferring an excited electron as an electron acceptor having large electronegativity due to excellent electron-retention properties of the molecule itself when accepting light in a mixed state with an electron acceptor even though not having a negative charge or an unshared electron pair.

In the present specification, the electron acceptor means those accepting electrons from the electron donor.

Examples of the substituents will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, the term “substituted or unsubstituted” means being substituted with one or two or more substituents selected from the group consisting of deuterium; a halogen group; a nitrile group; a nitro group; an imide group; an amide group; a carbonyl group; an ester group; a hydroxyl group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; and a substituted or unsubstituted heterocyclic group or being substituted with a substituent to which two or more substituents are linked among the substituents exemplified above, or having no substituent. For example, “the substituent to which two or more substituents are linked” may be a biphenyl group. That is, the biphenyl group may also be an aryl group, and may be interpreted as a substituent to which two phenyl groups are linked.

In the present specification, a halogen group may be fluorine, chlorine, bromine or iodine.

In the present specification, the number of carbon atoms of an imide group is not particularly limited, but is preferably 1 to 30. Specifically, the imide group may be a compound having the following structures, but is not limited thereto.

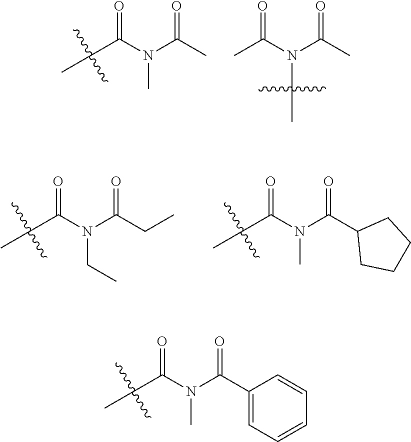

In the present specification, for an amide group, the nitrogen of the amide group may be substituted with hydrogen, a straight-chained, branched, or cyclic alkyl group having 1 to 30 carbon atoms, or an aryl group having 6 to 30 carbon atoms. Specifically, the amide group may be a compound having the following structural formulae, but is not limited thereto.

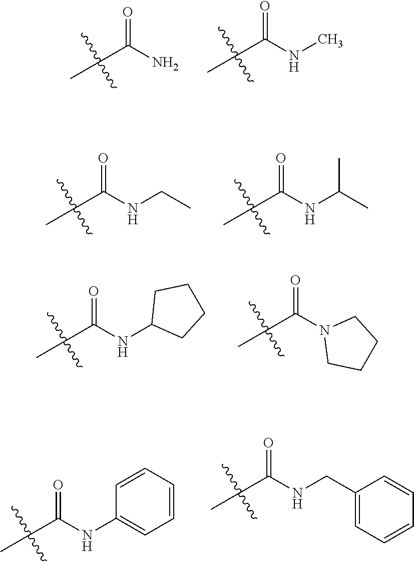

In the present specification, the number of carbon atoms of a carbonyl group is not particularly limited, but is preferably 1 to 30. Specifically, the carbonyl group may be a compound having the following structures, but is not limited thereto.

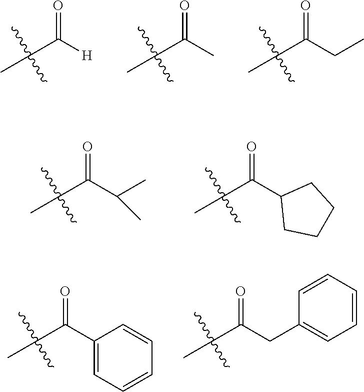

In the present specification, for an ester group, the oxygen of the ester group may be substituted with a straight-chained, branched, or cyclic alkyl group having 1 to 25 carbon atoms, or an aryl group having 6 to 30 carbon atoms. Specifically, the ester group may be a compound having the following structural formulae, but is not limited thereto.

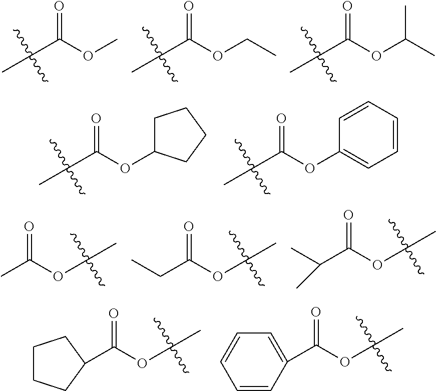

In the present specification, the alkyl group may be straight-chained or branched, and the number of carbon atoms thereof is not particularly limited, but is preferably 1 to 30. Specific examples thereof include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present specification, a cycloalkyl group is not particularly limited, but has preferably 3 to 30 carbon atoms, and specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present specification, the alkoxy group may be straight-chained, branched, or cyclic. The number of carbon atoms of the alkoxy group is not particularly limited, but is preferably 1 to 30. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, an amine group may be selected from the group consisting of —NH₂; an alkylamine group; an N-alkylarylamine group; an arylamine group; an N-arylheteroarylamine group; an N-alkylheteroarylamine group; and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, an N-phenylnaphthylamine group, a ditolylamine group, an N-phenyltolylamine group, a triphenylamine group, an N-phenylbiphenylamine group; an N-phenylnaphthylamine group; an N-biphenylnaphthylamine group; an N-naphthylfluorenylamine group; an N-phenylphenanthrenylamine group; an N-biphenylphenanthrenylamine group; an N-phenylfluorenylamine group; an N-phenyl terphenylamine group; an N-phenanthrenylfluorenylamine, group; an N-biphenylfluorenylamine group, and the like, but are not limited thereto.

In the present specification, an N-alkylarylamine group means an amine group in which an alkyl group and an aryl group are substituted with N of the amine group.

In the present specification, an N-arylheteroarylamine group means an amine group in which an aryl group and a heteroaryl group are substituted with N of the amine group.

In the present specification, an N-alkylheteroarylamine group means an amine group in which an alkyl group and a heteroaryl group are substituted with N of the amine group.

In the present specification, the alkyl group in the alkylamine group, the N-arylalkylamine group, the alkylthioxy group, the alkylsulfoxy group, and the N-alkylheteroarylamine group is the same as the above-described examples of the alkyl group. Specifically, examples of the alkylthioxy group include a methylthioxy group, an ethylthioxy group, a tert-butylthioxy group, a hexylthioxy group, an octylthioxy group, and the like, and examples of the alkylsulfoxy group include mesyl, an ethylsulfoxy group, a propylsulfoxy group, a butylsulfoxy group, and the like, but the examples are not limited thereto.

In the present specification, the alkenyl group may be straight-chained or branched, and the number of carbon atoms thereof is not particularly limited, but is preferably 2 to 30. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, specific examples of a silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be —BR₁₀₀R₁₀₁, and R₁₀₀ and R₁₀₁ are the same as or different from each other, and may be each independently selected from the group consisting of hydrogen; deuterium; halogen; a nitrile group; a substituted or unsubstituted monocyclic or polycyclic cycloalkyl group having 3 to 30 carbon atoms; a substituted or unsubstituted straight-chained or branched alkyl group having 1 to 30 carbon atoms; a substituted or unsubstituted monocyclic or polycyclic aryl group having 6 to 30 carbon atoms; and a substituted or unsubstituted monocyclic or polycyclic heteroaryl group having 2 to 30 carbon atoms.

In the present specification, specific examples of a phosphine oxide group include a diphenylphosphine oxide group, a dinaphthylphosphine oxide group, and the like, but are not limited thereto.

In the present specification, an aryl group is not particularly limited, but has preferably 6 to 30 carbon atoms, and the aryl group may be monocyclic or polycyclic.

When the aryl group is a monocyclic aryl group, the number of carbon atoms thereof is not particularly limited, but is preferably 6 to 30. Specific examples of the monocyclic aryl group include a phenyl group, a biphenyl group, a terphenyl group, and the like, but are not limited thereto.

When the aryl group is a polycyclic aryl group, the number of carbon atoms thereof is not particularly limited, but is preferably 10 to 30. Specific examples of the polycyclic aryl group include a naphthyl group, an anthracenyl group, a phenanthryl group, a triphenyl group, a pyrenyl group, a phenalenyl group, a perylenyl group, a chrysenyl group, a fluorenyl group, a fluoranthenyl group, and the like, but are not limited thereto.

In the present specification, the fluorenyl group may be substituted, and adjacent substituents may be bonded to each other to form a ring.

When the fluorenyl group is substituted, the substituent may be

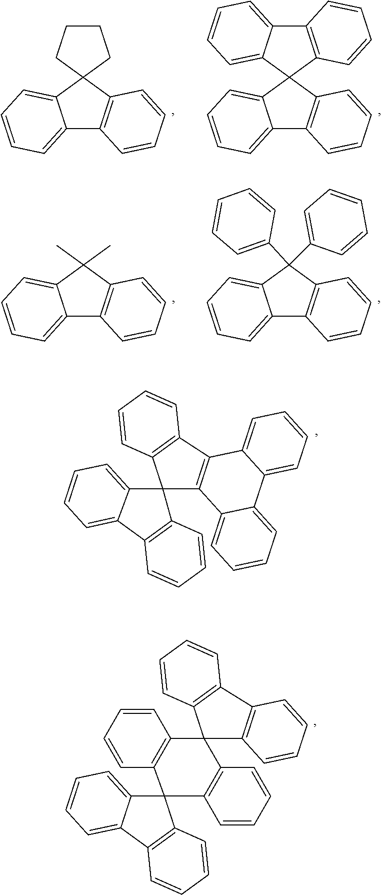

and the like. However, the substituent is not limited thereto.

In the present specification, the “adjacent” group may mean a substituent substituted with an atom directly linked to an atom in which the corresponding substituent is substituted, a substituent disposed sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other.

In the present specification, the aryl group in the aryloxy group, the arylthioxy group, the arylsulfoxy group, the N-arylalkylamine group, the N-arylheteroarylamine group, and the arylphosphine group is the same as the above-described examples of the aryl group. Specifically, examples of the aryloxy group include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group, and the like, examples of the arylthioxy group include a phenylthioxy group, a 2-methylphenylthioxy group, a 4-tert-butylphenylthioxy group, and the like, and examples of the arylsulfoxy group include a benzenesulfoxy group, a p-toluenesulfoxy group, and the like, but the examples are not limited thereto.

In the present specification, examples of an arylamine group include a substituted or unsubstituted monoarylamine group or a substituted or unsubstituted diarylamine group. The aryl group in the arylamine group may be a monocyclic aryl group or a polycyclic aryl group. The arylamine group including two or more aryl groups may include a monocyclic aryl group, a polycyclic aryl group, or both a monocyclic aryl group and a polycyclic aryl group. For example, the aryl group in the arylamine group may be selected from the above-described examples of the aryl group.

In the present specification, a heteroaryl group includes one or more atoms other than carbon, that is, one or more heteroatoms, and specifically, the heteroatom may include one or more atoms selected from the group consisting of O, N, Se, S, and the like. The number of carbon atoms thereof is not particularly limited, but is preferably 2 to 30, and the heteroaryl group may be monocyclic or polycyclic. Examples of a heterocyclic group include a thiophene group, a furanyl group, a pyrrole group, an imidazolyl group, a thiazolyl group, an oxazolyl group, an oxadiazolyl group, a pyridyl group, a bipyridyl group, a pyrimidyl group, a triazinyl group, a triazolyl group, an acridyl group, a pyridazinyl group, a pyrazinyl group, a qinolinyl group, a quinazolyl group, a quinoxalinyl group, a phthalazinyl group, a pyridopyrimidyl group, a pyridopyrazinyl group, a pyrazinopyrazinyl group, an isoquinolyl group, an indolyl group, a carbazolyl group, a benzoxazolyl group, a benzimidazolyl group, a benzothiazolyl group, a benzocarbazolyl group, a benzothiophene group, a dibenzothiophene group, a benzofuranyl group, a phenanthrolinyl group (phenanthroline), an isoxazolyl group, a thiadiazolyl group, a phenothiazinyl group, a dibenzofuranyl group, and the like, but are not limited thereto.

In the present specification, examples of a heteroarylamine group include a substituted or unsubstituted monoheteroarylamine group or a substituted or unsubstituted diheteroarylamine group. The heteroarylamine group including two or more heteroaryl groups may include a monocyclic heteroaryl group, a polycyclic heteroaryl group, or both a monocyclic heteroaryl group and a polycyclic heteroaryl group. For example, the heteroaryl group in the heteroarylamine group may be selected from the above-described examples of the heteroaryl group.

In the present specification, examples of the heteroaryl group in the N-arylheteroarylamine group and the N-alkylheteroarylamine group are the same as the above-described examples of the heteroaryl group.

According to an exemplary embodiment of the present specification, in Formula 1, Ar1 is a monocyclic or polycyclic aryl group.

According to an exemplary embodiment of the present specification, in Formula 1, R1 and R2 are hydrogen.

According to an exemplary embodiment of the present specification, in Formula 1, Ar1 is a substituted or unsubstituted monocyclic or polycyclic aryl group having 6 to 30 carbon atoms.

According to an exemplary embodiment of the present specification, in Formula 1, Ar1 is a monocyclic or polycyclic aryl group having 6 to 30 carbon atoms.

According to an exemplary embodiment of the present specification, in Formula 1, Ar1 is a monocyclic aryl group having 6 to 12 carbon atoms.

According to an exemplary embodiment of the present specification, in Formula 1, Ar1 is a phenyl group.

According to an exemplary embodiment of the present specification, in Formula 1, Ar2 and Ar3 are the same as or different from each other, and are each independently a straight-chained or branched alkyl group.

According to an exemplary embodiment of the present specification, in Formula 1, Ar2 and Ar3 are the same as or different from each other, and are each independently a substituted or unsubstituted straight-chained or branched alkyl group having 1 to 30 carbon atoms.

According to an exemplary embodiment of the present specification, in Formula 1, Ar2 and Ar3 are the same as or different from each other, and are each independently a straight-chained or branched alkyl group having 1 to 30 carbon atoms.

According to an exemplary embodiment of the present specification, in Formula 1, Ar2 and Ar3 are the same as or different from each other, and are each independently a straight-chained alkyl group having 1 to 10 carbon atoms.

According to an exemplary embodiment of the present specification, in Formula 1, Ar2 and Ar3 are a methyl group.

According to an exemplary embodiment of the present specification, in Formula 1, L1 and L2 are the same as or different from each other, and are each independently selected from the following Formulae A to C.

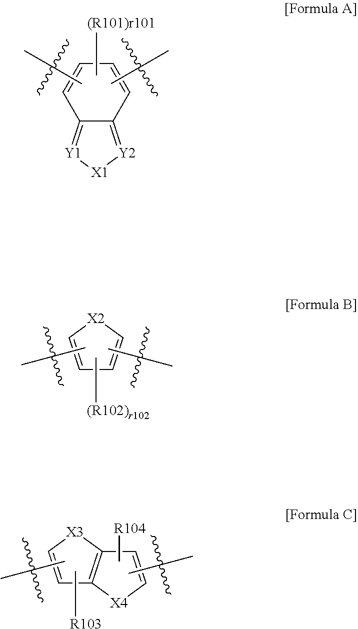

In Formulae A to C,

X1 to X4 are the same as or different from each other, and are each independently O, S, or Se,

Y1 to Y2 are the same as or different from each other, and are each independently N or P,

R101 to R104 are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heteroaryl group, r101 and r102 are each 1 or 2,

when r101 is 2, a plurality of R101's is the same as or different from each other,

when r101 is 2, a plurality of R102's is the same as or different from each other, and

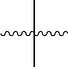

is a moiety bonded to Formula 1.

According to an exemplary embodiment of the present specification, in Formula A, X1 is S.

According to an exemplary embodiment of the present specification, in Formula A, X1 is Se.

According to an exemplary embodiment of the present specification, in Formula A, Y1 and Y2 are N.

According to an exemplary embodiment of the present specification, in Formula A, R101 is hydrogen.

According to an exemplary embodiment of the present specification, in Formula B, X2 is S.

According to an exemplary embodiment of the present specification, in Formula B, R102 is hydrogen.

According to an exemplary embodiment of the present specification, in Formula C, X3 and X4 are S.

According to an exemplary embodiment of the present specification, in Formula C, R103 and R104 are hydrogen.

According to an exemplary embodiment of the present specification, in Formula 1, L1 is a group represented by Formula A.

According to an exemplary embodiment of the present specification, in Formula 1, L1 is a group represented by Formula B.

According to an exemplary embodiment of the present specification, in Formula 1, L1 is a group represented by Formula C.

According to an exemplary embodiment of the present specification, in Formula 1, L2 is a group represented by Formula A.

According to an exemplary embodiment of the present specification, in Formula 1, L2 is a group represented by Formula B.

According to an exemplary embodiment of the present specification, in Formula 1, L2 is a group represented by Formula C.

According to an exemplary embodiment of the present specification, L1 and L2 are the same as or different from each other, and are each independently a substituted or unsubstituted divalent benzothiadiazole group; a substituted or unsubstituted divalent thiophene group; or a substituted or unsubstituted divalent thienothiophene group.

According to an exemplary embodiment of the present specification, L1 and L2 are the same as or different from each other, and are each independently a divalent benzothiadiazole group; a divalent thiophene group; or a divalent thienothiophene group.

According to an exemplary embodiment of the present specification, in Formula 1, L1 is a divalent benzothiadiazole group.

According to an exemplary embodiment of the present specification, in Formula 1, L1 is a divalent thiophene group.

According to an exemplary embodiment of the present specification, in Formula 1, L1 is a divalent thienothiophene group.

According to an exemplary embodiment of the present specification, in Formula 1, L2 is a divalent benzothiadiazole group.

According to an exemplary embodiment of the present specification, in Formula 1, L2 is a divalent thiophene group.

According to an exemplary embodiment of the present specification, in Formula 1, L2 is a divalent thienothiophene group.

According to an exemplary embodiment of the present specification, Formula 1 is represented by any one of the following Formulae 1-1 to 1-4.

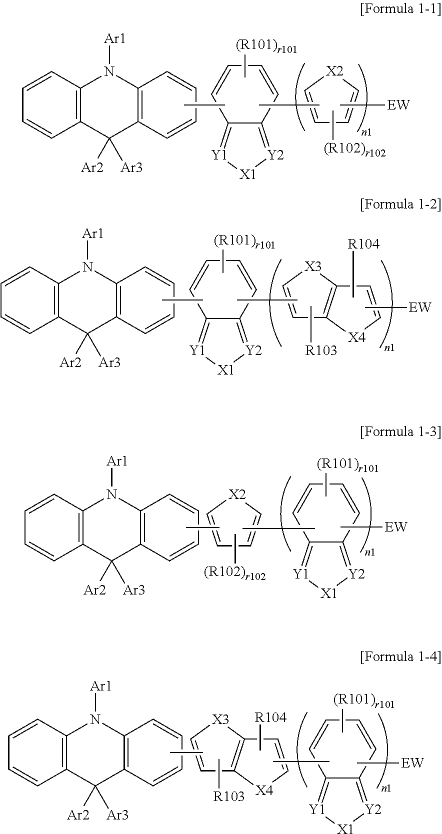

In Formulae 1-1 to 1-4,

the definitions of Ar1 to Ar3, n1, and EW are the same as those defined in Formula 1,

X1 to X4 are the same as or different from each other, and are each independently O, S, or Se,

Y1 and Y2 are the same as or different from each other, and are each independently N or P,

R101 to R104 are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heteroaryl group,

r101 and r102 are each 1 or 2,

when r101 is 2, a plurality of R101's is the same as or different from each other, and

when r102 is 2, a plurality of R102's is the same as or different from each other.

According to an exemplary embodiment of the present specification, Formula 1 is represented by any one of the following Formulae 1-5 to 1-14.

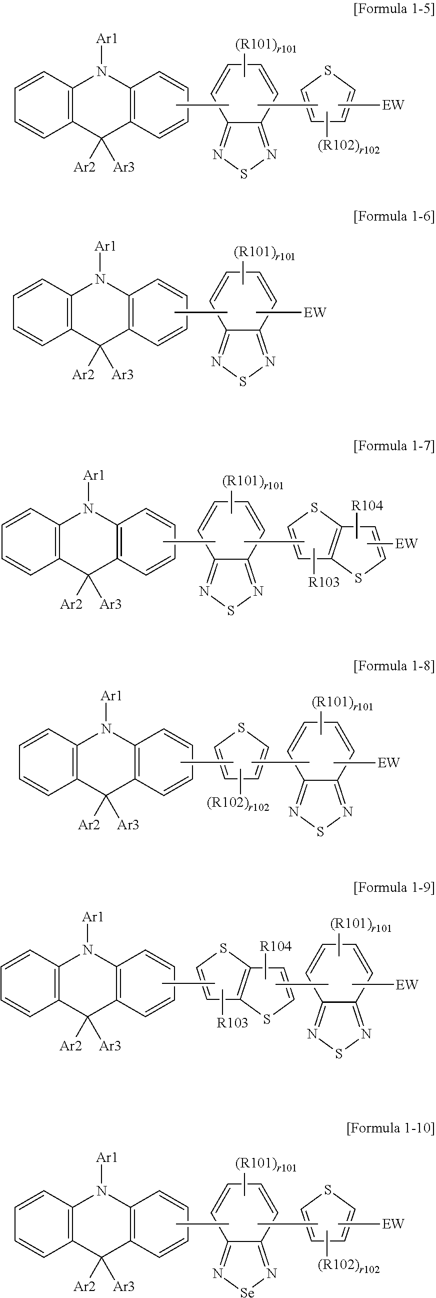

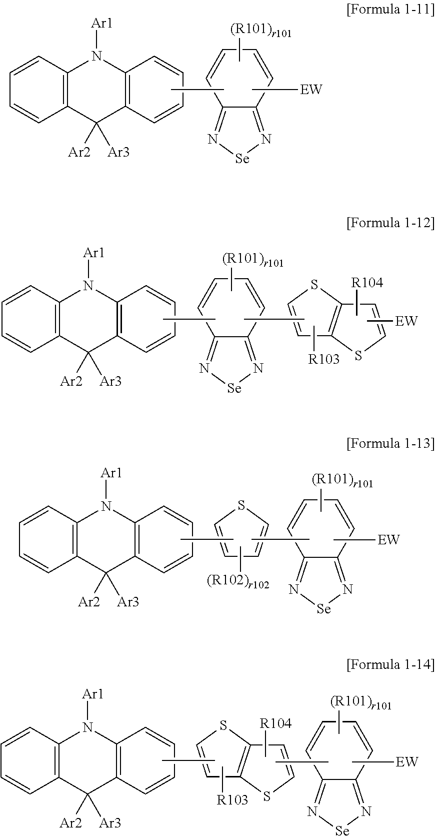

In Formulae 1-5 to 1-14, the definitions of Ar1 to Ar3 and EW are the same as those defined in Formula 1,

R101 to R104 are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heteroaryl group,

r101 and r102 are each 1 or 2,

when r101 is 2, a plurality of R101's is the same as or different from each other, and when r102 is 2, a plurality of R102's is the same as or different from each other.

According to an exemplary embodiment of the present specification, in Formula 1,

EW is selected from the following structures.

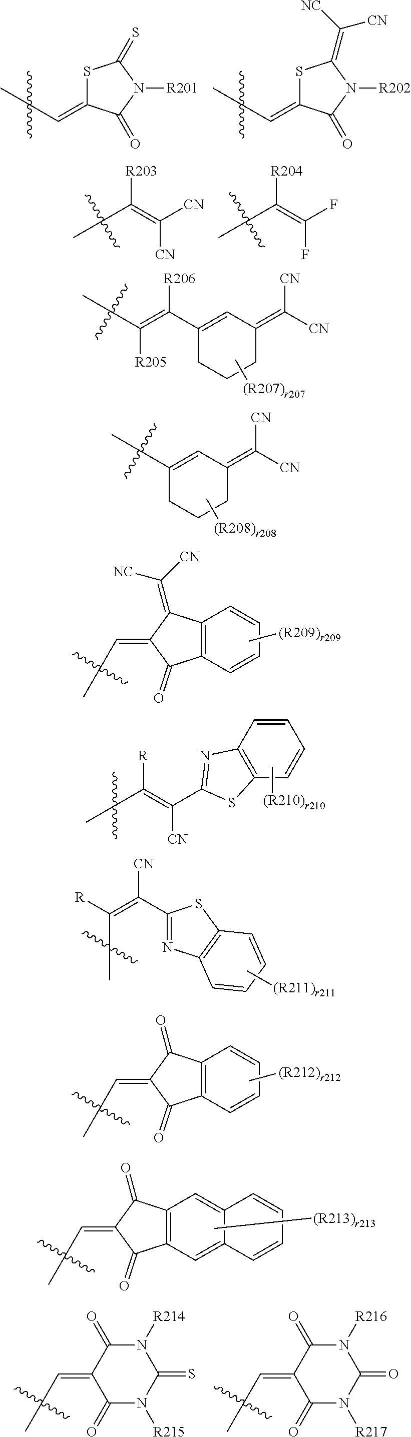

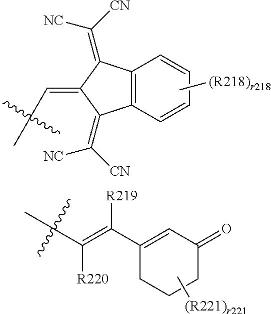

In the structures,

R and R201 to R221 are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heteroaryl group,

r207, r208, and r221 are each an integer from 1 to 7,

r209, r210, r211, r212, and r218 are each an integer from 1 to 4,

r213 is an integer from 1 to 6,

when r207 is 2 or more, a plurality of R207's is the same as or different from each other,

when r208 is 2 or more, a plurality of R208's is the same as or different from each other,

when r209 is 2 or more, a plurality of R209's is the same as or different from each other,

when r210 is 2 or more, a plurality of R210's is the same as or different from each other,

when r211 is 2 or more, a plurality of R211's is the same as or different from each other,

when r212 is 2 or more, a plurality of R212's is the same as or different from each other,

when r213 is 2 or more, a plurality of R213's is the same as or different from each other,

when r218 is 2 or more, a plurality of R218's is the same as or different from each other,

when r221 is 2 or more, a plurality of R221's is the same as or different from each other, and

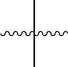

is a moiety bonded to Formula 1.

According to an exemplary embodiment of the present specification, in Formula 1, EW is selected from the following structures.

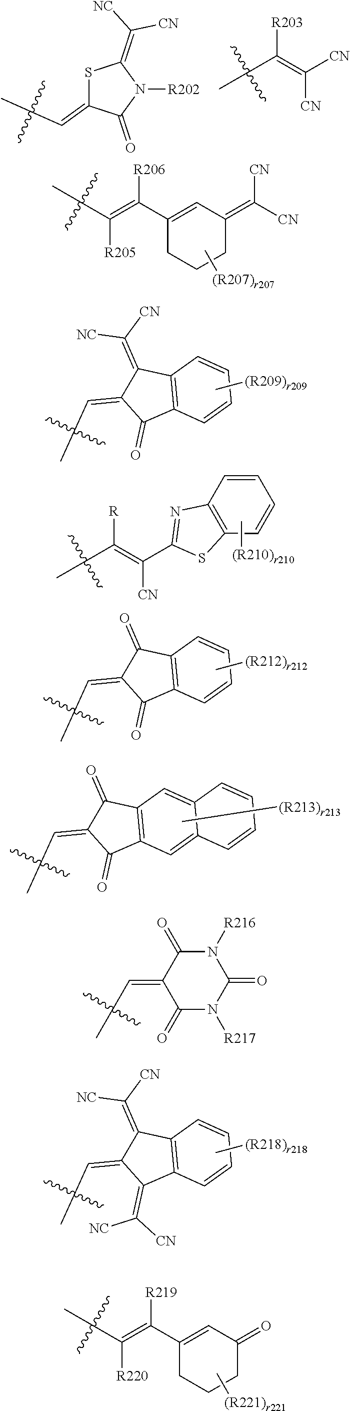

In the structures,

R, R202, R203, R205 to R207, R209, R210, R212, R213, and R216 to R221 are the same as or different from each other, and are each independently hydrogen; a substituted or unsubstituted alkyl group; or a substituted or unsubstituted aryl group,

r207 and r221 are each an integer from 1 to 7,

r209, r210, r212, and r218 are each an integer from 1 to 4,

r213 is an integer from 1 to 6,

when r207 is 2 or more, a plurality of R207's is the same as or different from each other,

when r209 is 2 or more, a plurality of R209's is the same as or different from each other,

when r210 is 2 or more, a plurality of R210's is the same as or different from each other,

when r212 is 2 or more, a plurality of R212's is the same as or different from each other,

when r213 is 2 or more, a plurality of R213's is the same as or different from each other,

when r218 is 2 or more, a plurality of R218's is the same as or different from each other,

when r221 is 2 or more, a plurality of R221's is the same as or different from each other, and

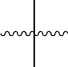

is a moiety bonded to Formula 1.

According to an exemplary embodiment of the present specification, R is hydrogen.

According to another exemplary embodiment of the present specification, R202 is a substituted or unsubstituted straight-chained or branched alkyl group having 1 to 30 carbon atoms.

According to another exemplary embodiment of the present specification, R202 is a straight-chained or branched alkyl group having 1 to 30 carbon atoms.

According to another exemplary embodiment of the present specification, R202 is a straight-chained alkyl group having 1 to 10 carbon atoms.

According to another exemplary embodiment of the present specification, R202 is a methyl group.

According to another exemplary embodiment of the present specification, R203 is hydrogen.

According to another exemplary embodiment of the present specification, R205 and R206 are hydrogen.

According to another exemplary embodiment of the present specification, R207 is hydrogen; or a substituted or unsubstituted straight-chained or branched alkyl group having 1 to 30 carbon atoms.

According to another exemplary embodiment of the present specification, R207 is hydrogen; or a straight-chained or branched alkyl group having 1 to 30 carbon atoms.

According to another exemplary embodiment of the present specification, R207 is hydrogen; or a straight-chained alkyl group having 1 to 10 carbon atoms.

According to another exemplary embodiment of the present specification, R207 is hydrogen; or a methyl group.

According to another exemplary embodiment of the present specification, R209 is hydrogen.

According to another exemplary embodiment of the present specification, R210 is hydrogen.

According to another exemplary embodiment of the present specification, R212 is hydrogen.

According to another exemplary embodiment of the present specification, R213 is hydrogen.

According to another exemplary embodiment of the present specification, R216 and R217 are the same as or different from each other, and are each independently a substituted or unsubstituted straight-chained or branched alkyl group having 1 to 30 carbon atoms.

According to yet another exemplary embodiment of the present specification, R216 and R217 are the same as or different from each other, and are each independently a straight-chained or branched alkyl group having 1 to 30 carbon atoms.

According to another exemplary embodiment of the present specification, R216 and R217 are the same as or different from each other, and are each independently a straight-chained alkyl group having 1 to 10 carbon atoms.

According to another exemplary embodiment of the present specification, R216 and R217 are a methyl group.

According to another exemplary embodiment of the present specification, R218 is hydrogen.

According to another exemplary embodiment of the present specification, R219 is hydrogen; or a substituted or unsubstituted straight-chained or branched alkyl group having 1 to 30 carbon atoms.

According to another exemplary embodiment of the present specification, R219 is hydrogen; or a straight-chained or branched alkyl group having 1 to 30 carbon atoms.

According to another exemplary embodiment of the present specification, R219 is hydrogen; or a straight-chained alkyl group having 1 to 10 carbon atoms.

According to another exemplary embodiment of the present specification, R219 is hydrogen; or a methyl group.

According to an exemplary embodiment of the present specification, in Formula 1, EW is selected from the following structures.

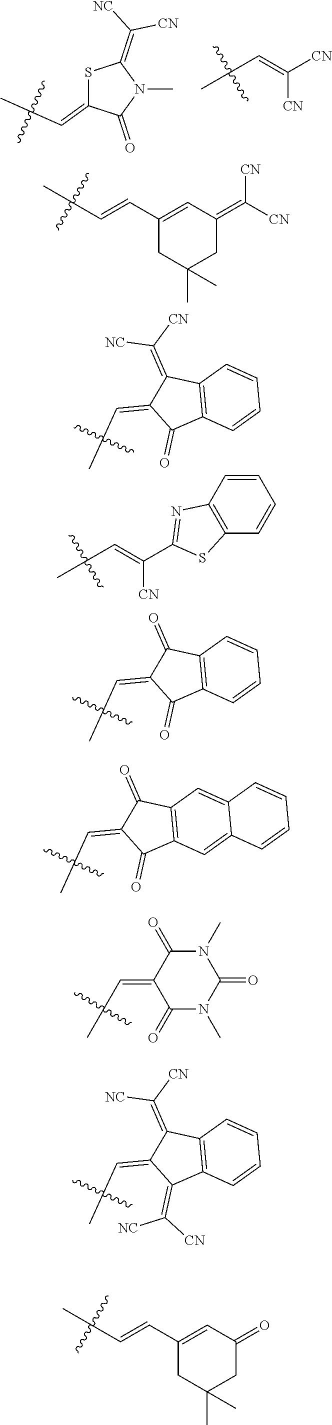

In the structures,

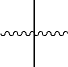

is a moiety bonded to Formula 1.

According to an exemplary embodiment of the present specification, Formula 1 is selected from the following compounds.

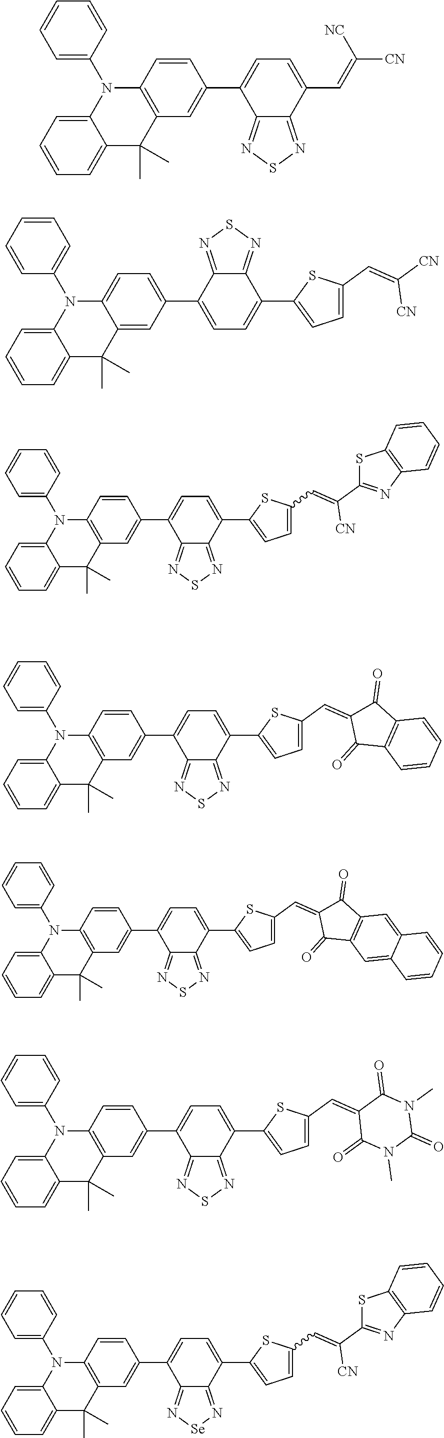

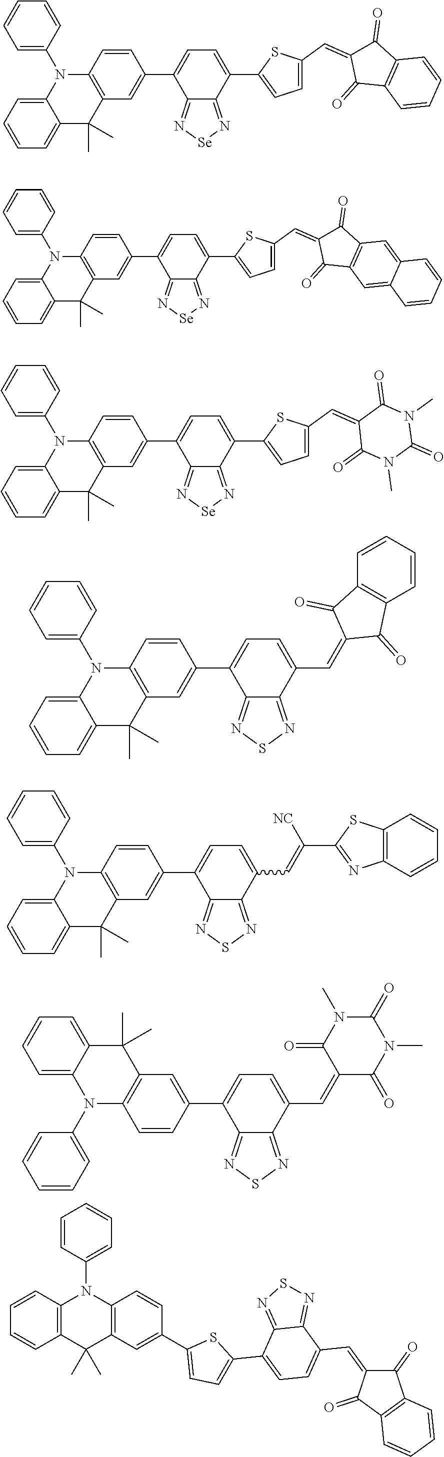

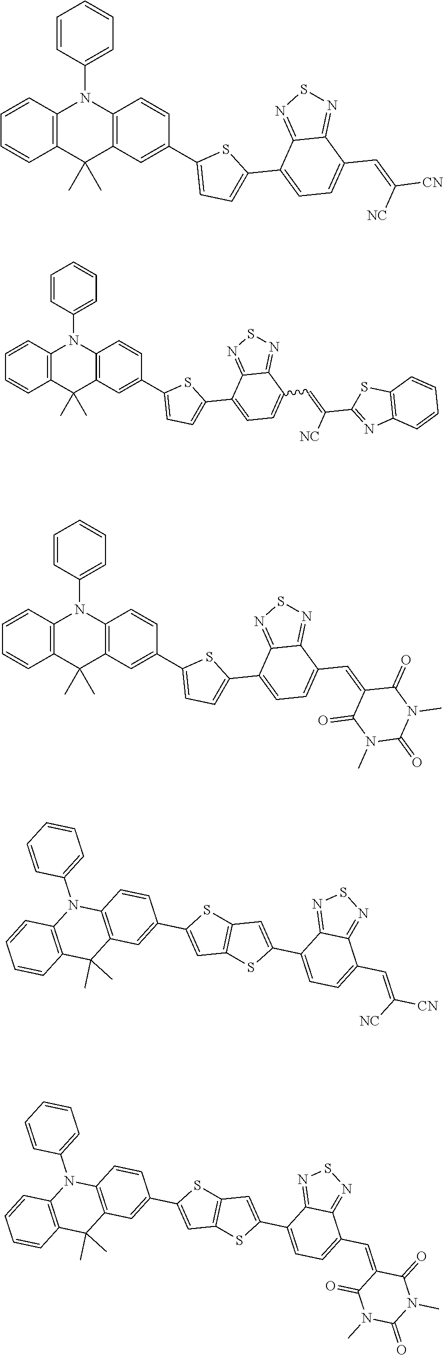 [SMILES: CC1(C)c2ccccc2N(c2ccccc2)c2ccc(-c3ccc(-c4ccc(C=C(C#N)C#N)c5nsnc45)s3)cc21 ; CC1(C)c2ccccc2N(c2ccccc2)c2ccc(-c3ccc(-c4ccc(C=C(C#N)c5nc6ccccc6s5)c5nsnc45)s3)cc21 ; CN1C(=O)C(=Cc2ccc(-c3ccc(-c4ccc5c(c4)C(C)(C)c4ccccc4N5c4ccccc4)s3)c3nsnc23)C(=O)N(C)C1=O ; CC1(C)c2ccccc2N(c2ccccc2)c2ccc(-c3cc4sc(-c5ccc(C=C(C#N)C#N)c6nsnc56)cc4s3)cc21 ; CN1C(=O)C(=Cc2ccc(-c3cc4sc(-c5ccc6c(c5)C(C)(C)c5ccccc5N6c5ccccc5)cc4s3)c3nsnc23)C(=O)N(C)C1=O]

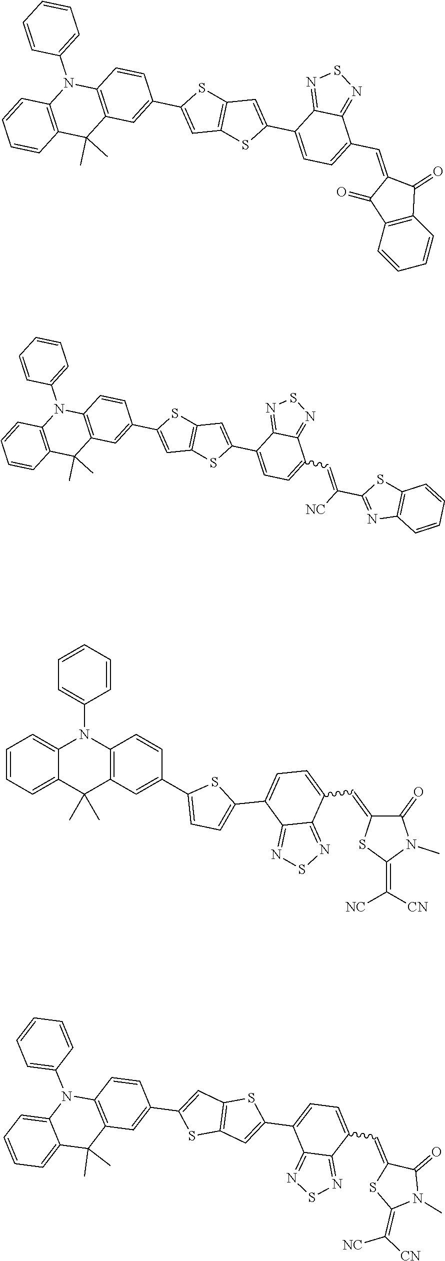 [SMILES: CC1(C)c2ccccc2N(c2ccccc2)c2ccc(-c3cc4sc(-c5ccc(C=C6C(=O)c7ccccc7C6=O)c6nsnc56)cc4s3)cc21 ; CC1(C)c2ccccc2N(c2ccccc2)c2ccc(-c3cc4sc(-c5ccc(C=C(C#N)c6nc7ccccc7s6)c6nsnc56)cc4s3)cc21 ; Cn1c(=C(C#N)C#N)sc(=Cc2ccc(-c3ccc(-c4ccc5c(c4)C(C)(C)c4ccccc4N5c4ccccc4)s3)c3nsnc23)c1=O ; Cn1c(=C(C#N)C#N)sc(=Cc2ccc(-c3cc4sc(-c5ccc6c(c5)C(C)(C)c5ccccc5N6c5ccccc5)cc4s3)c3nsnc23)c1=O]

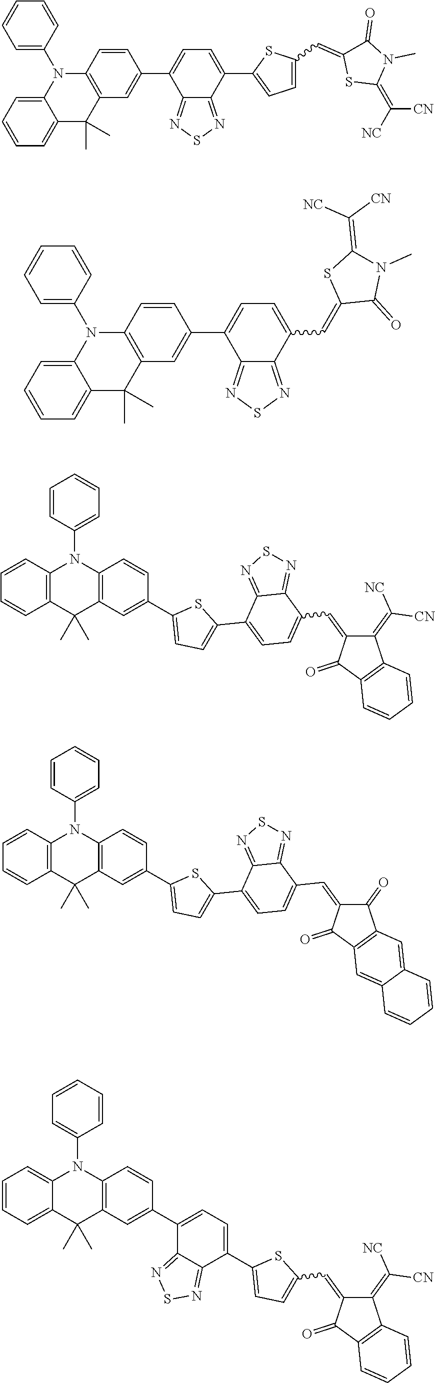 [SMILES: Cn1c(=C(C#N)C#N)sc(=Cc2ccc(-c3ccc(-c4ccc5c(c4)C(C)(C)c4ccccc4N5c4ccccc4)c4nsnc34)s2)c1=O ; Cn1c(=C(C#N)C#N)sc(=Cc2ccc(-c3ccc4c(c3)C(C)(C)c3ccccc3N4c3ccccc3)c3nsnc23)c1=O ; CC1(C)c2ccccc2N(c2ccccc2)c2ccc(-c3ccc(-c4ccc(C=C5C(=O)c6ccccc6C5=C(C#N)C#N)c5nsnc45)s3)cc21 ; CC1(C)c2ccccc2N(c2ccccc2)c2ccc(-c3ccc(-c4ccc(C=C5C(=O)c6cc7ccccc7cc6C5=O)c5nsnc45)s3)cc21 ; CC1(C)c2ccccc2N(c2ccccc2)c2ccc(-c3ccc(-c4ccc(C=C5C(=O)c6ccccc6C5=C(C#N)C#N)s4)c4nsnc34)cc21]

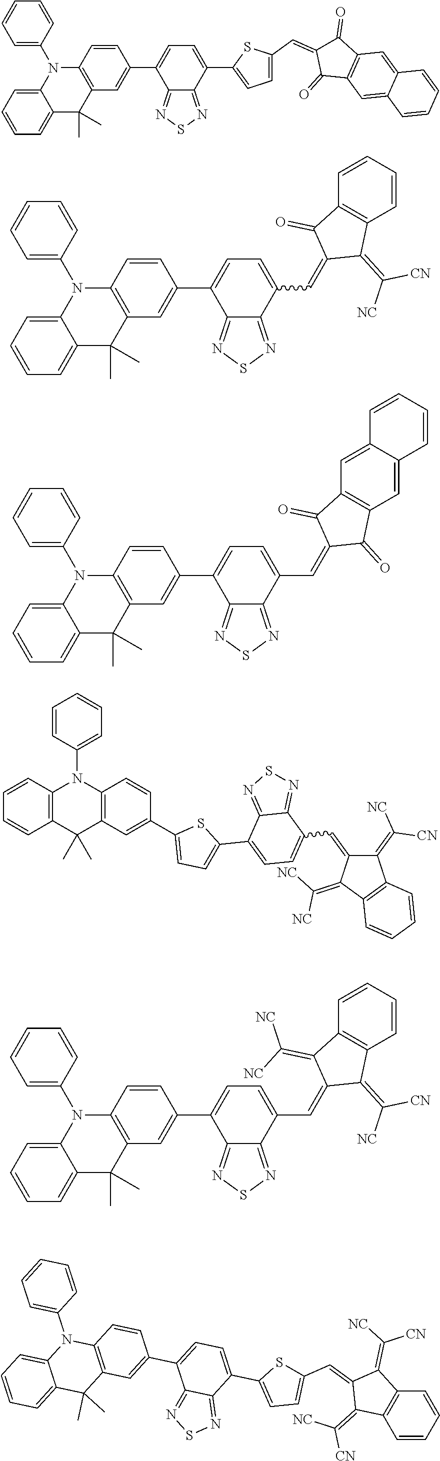

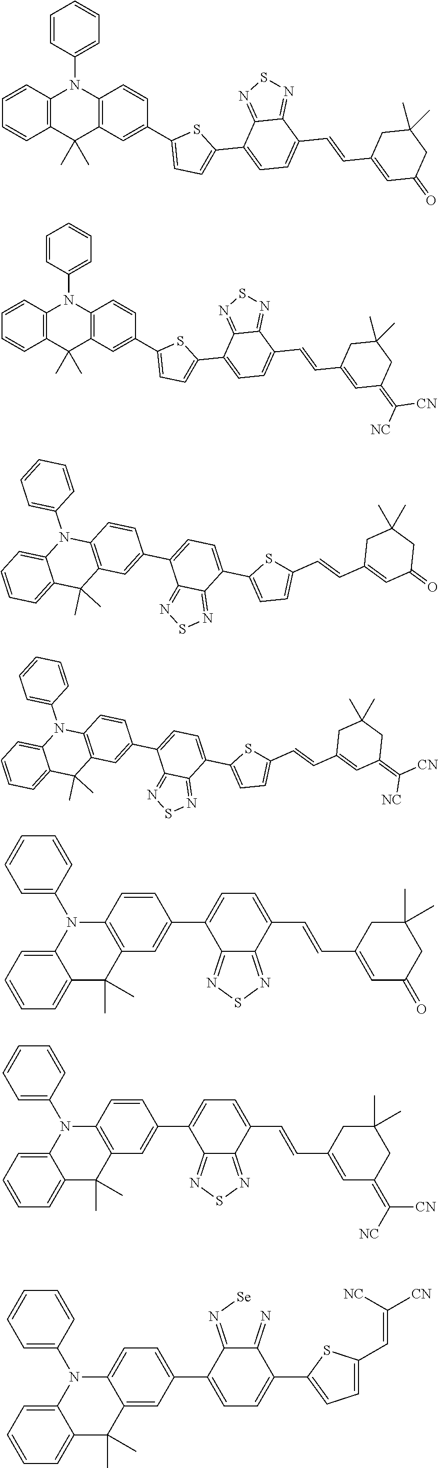

In the compounds, [symbol] means that isomers having a trans structure and a cis structure are mixed with each other.

An exemplary embodiment of the present specification provides an organic photoelectric device including: a first electrode; a second electrode disposed to face the first electrode; and an organic material layer having one or more layers disposed between the first electrode and the second electrode, in which one or more layers of the organic material layer include the heterocyclic compound.

The organic photoelectric device according to an exemplary embodiment of the present specification includes a first electrode, a photoactive layer, and a second electrode. The organic photoelectric device may further include a substrate, a hole transport layer, and/or an electron transport layer.

According to an exemplary embodiment of the present specification, the organic photoelectric device may further include an additional organic material layer. The organic photoelectric device may reduce the number of organic material layers by using an organic material which simultaneously has various functions.

According to an exemplary embodiment of the present specification, the first electrode is an anode, and the second electrode is a cathode. In another exemplary embodiment, the first electrode is a cathode, and the second electrode is an anode.

According to an exemplary embodiment of the present specification, in the organic photoelectric device, a cathode, a photoactive layer, and an anode may be arranged in this order, and an anode, a photoactive layer, and a cathode may be arranged in this order, but the arrangement order is not limited thereto.

In another exemplary embodiment, in the organic photoelectric device, an anode, a hole transport layer, a photoactive layer, an electron transport layer, and a cathode may also be arranged in this order, and a cathode, an electron transport layer, a photoactive layer, a hole transport layer, and an anode may also be arranged in this order, but the arrangement order is not limited thereto.

According to an exemplary embodiment of the present specification, the organic photoelectric device has a normal structure. In the normal structure, a substrate, an anode, an organic material layer including a photoactive layer, and a cathode may be stacked in this order.

According to an exemplary embodiment of the present specification, the organic photoelectric device has an inverted structure. In the inverted structure, a substrate, a cathode, an organic material layer including a photoactive layer, and an anode may be stacked in this order.

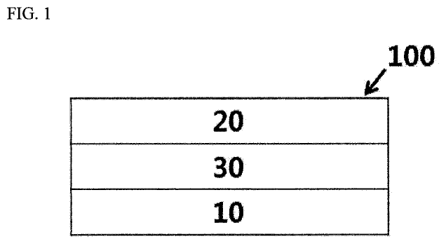

FIG. 1 is a view illustrating an organic photoelectric device 100 according to an exemplary embodiment of the present specification, and according to FIG. 1, in the organic photoelectric device 100, light is incident from the sides of a first electrode 10 and/or a second electrode 20, so that when an active layer 30 absorbs light in the entire wavelength regions, excitons may be produced therein. The exciton is separated into a hole and an electron in the active layer 30, the separated hole moves to an anode side which is one of the first electrode 10 and the second electrode 20, and the separated electron moves to a cathode side which is the other of the first electrode 10 and the second electrode 20, so that an electric current may flow in the organic photoelectric device.

According to an exemplary embodiment of the present specification, the organic photoelectric device has a tandem structure.

According to an exemplary embodiment of the present specification, the organic material layer includes a photoactive layer, the photoactive layer has a bilayer thin film structure including an n-type organic material layer and a p-type organic material layer, and the p-type organic material layer includes the heterocyclic compound.

According to an exemplary embodiment of the present specification, the organic material layer includes a photoactive layer, the photoactive layer includes an electron donor material and an electron acceptor material, and the electron donor material includes the heterocyclic compound.

According to an exemplary embodiment of the present specification, the electron acceptor material and the n-type organic material layer may be selected from the group consisting of fullerene, fullerene derivatives, bathocuproine, semi-conducting elements, semi-conducting compounds, and combinations thereof. Specifically, the electron acceptor material and the n-type organic material layer are one or two or more compounds selected from the group consisting of fullerene, fullerene derivatives ((6,6)-phenyl-C61-butyric acid-methylester (PCBM) or (6,6)-phenyl-C61-butyric acid-cholesteryl ester (PCBCR)), perylene, polybenzimidazole (PBI), and 3,4,9,10-perylene-tetracarboxylic bis-benzimidazole (PTCBI).

According to an exemplary embodiment of the present specification, the electron donor and the electron acceptor constitute a bulk heterojunction (BHJ).

The bulk heterojunction means that an electron donor material and an electron acceptor material are mixed with each other in a photoactive layer.

In the organic photoelectric device according to an exemplary embodiment of the present specification, materials and/or methods in the art may be used without limitation, except that the heterocyclic compound represented by Formula 1 is used as a photoactive layer of the organic photoelectric device.

In the present specification, the substrate may be a glass substrate or a transparent plastic substrate having excellent transparency, surface smoothness, ease of handling, and waterproofing properties, but is not limited thereto, and the substrate is not limited as long as the substrate is typically used in the organic solar cell. Specific examples thereof include glass or polyethylene terephthalate (PET), polyethylene naphthalate (PEN), polypropylene (PP), polyimide (PI), triacetyl cellulose (TAC), and the like, but are not limited thereto.

The anode electrode may be made of a material which is transparent and has excellent conductivity, but is not limited thereto. Examples thereof include: a metal, such as vanadium, chromium, copper, zinc, and gold, or an alloy thereof; a metal oxide, such as zinc oxide, indium oxide, indium tin oxide (ITO), and indium zinc oxide (IZO); a combination of metal and oxide, such as ZnO:Al or SnO₂:Sb; an electrically conductive polymer, such as poly(3-methylthiophene), poly[3,4-(ethylene-1,2-dioxy)thiophene] (PEDT), polypyrrole, and polyaniline, and the like, but are not limited thereto.

A method of forming the anode electrode is not particularly limited, but the anode electrode may be formed, for example, by being applied onto one surface of a substrate using a method such as sputtering, e-beam, thermal deposition, spin coating, screen printing, inkjet printing, doctor blade, or gravure printing, or by being coated in the form of a film.

When the anode electrode is formed on a substrate, the anode electrode may be subjected to processes of cleaning, removing moisture, and hydrophilic modification.

For example, a patterned ITO substrate is sequentially cleaned with a cleaning agent, acetone, and isopropyl alcohol (IPA), and then dried on a hot plate at 100° C. to 150° C. for 1 to 30 minutes, preferably at 120° C. for 10 minutes in order to remove moisture, and when the substrate is completely cleaned, the surface of the substrate is hydrophilically modified.

Through the surface modification as described above, the junction surface potential may be maintained at a level suitable for a surface potential of a photoactive layer. Further, during the modification, a polymer thin film may be easily formed on an anode electrode, and the quality of the thin film may also be improved.

Examples of a pre-treatment technology for an anode electrode include a) a surface oxidation method using a parallel flat plate-type discharge, b) a method of oxidizing a surface through ozone produced by using UV rays in a vacuum state, c) an oxidation method using oxygen radicals produced by plasma, and the like.

One of the methods may be selected depending on the state of the anode electrode or the substrate. However, commonly in all the methods, it is preferred to prevent oxygen from being separated from the surface of the anode electrode or the substrate, and maximally inhibit moisture and organic materials from remaining. In this case, it is possible to maximize a substantial effect of the pre-treatment.

As a specific example, it is possible to use a method of oxidizing the surface through ozone produced by using UV. In this case, a patterned ITO substrate after being ultrasonically cleaned is baked on a hot plate and dried well, and then introduced into a chamber, and the patterned ITO substrate may be cleaned by ozone generated by allowing an oxygen gas to react with UV light by operating a UV lamp.

However, the surface modification method of the patterned ITO substrate in the present specification need not be particularly limited, and any method may be used as long as the method is a method of oxidizing a substrate.

The cathode electrode may be a metal having a low work function, but is not limited thereto. Specific examples thereof include: a metal such as magnesium, calcium, sodium, potassium, titanium, indium, yttrium, lithium, gadolinium, aluminum, silver, tin, and lead, or an alloy thereof; and a multi-layer structured material such as LiF/Al, LiO₂/Al, LiF/Fe, Al:Li, Al:BaF₂, and Al:BaF₂:Ba, but are not limited thereto.

The cathode electrode may be deposited and formed in a thermal evaporator showing a vacuum degree of 5×10⁻⁷ torr or less, but is not limited to this method.

A material for the hole transport layer and/or a material for the electron transport layer serve to efficiently transfer electrons and holes separated from a photoactive layer to an electrode, and the materials are not particularly limited.

Examples of the material for the hole transport layer include: poly(3,4-ethylenedioxythiophene) doped with poly(styrenesulfonic acid) (PEDOT:PSS); molybdenum oxide (MoOₓ, 0<x=3); vanadium oxide (V₂O₅); nickel oxide (NiO); tungsten oxide (WOₓ, 0<x=3); and the like, but are not limited thereto.

Examples of the material for the electron transport layer include one selected from 1,4,5,8-naphthalene-tetracarboxylic dianhydride (NTCDA), bathocuproine (BCP), LiF, Alq₃, Gaq₃, Inq₃, Znq₂, Zn(BTZ)₂, BeBq₂, and a combination thereof, but are not limited thereto.

The photoactive layer may be formed by dissolving a photoactive material such as an electron donor material and/or an electron acceptor material in an organic solvent, and then applying the solution by a method such as spin coating, dip coating, screen printing, spray coating, doctor blade, and brush painting, but the forming method is not limited thereto.

The organic photoelectric device according to an exemplary embodiment of the present specification may be applied to a solar cell, an image sensor, a photodetector, a photosensor, a phototransistor, and the like, but the application range is not limited thereto.

An exemplary embodiment of the present specification provides an organic image sensor including the organic photoelectric device.

The organic image sensor according to an exemplary embodiment of the present specification may be applied to an electronic device, and may be applied to, for example, a mobile phone, a digital camera, and the like, but the application range is not limited thereto.

### MODE FOR INVENTION

A preparation method of the heterocyclic compound and the manufacture of an organic photoelectric device including the same will be described in detail in the following Preparation Examples and Examples. However, the following Examples are provided for exemplifying the present specification, and the scope of the present specification is not limited thereby.

### Preparation Example 1. Preparation of Compound 1

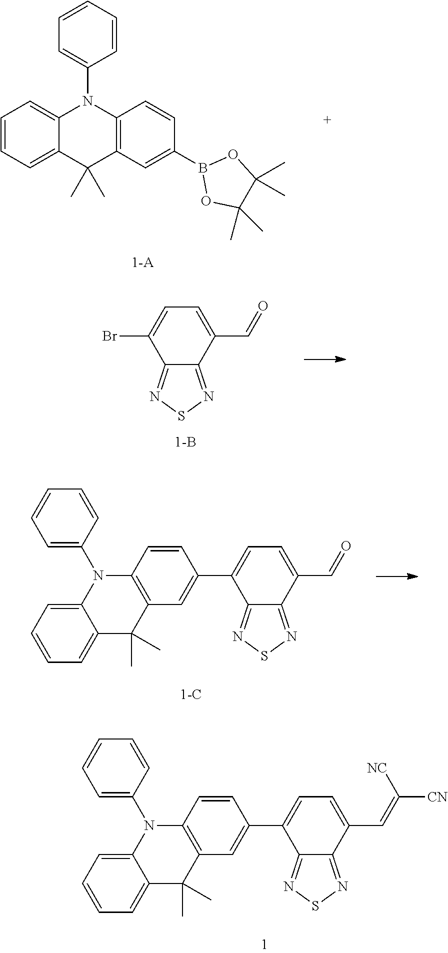 [SMILES: CC1(C)c2ccccc2N(c2ccccc2)c2ccc(B3OC(C)(C)C(C)(C)O3)cc21 ; O=Cc1ccc(Br)c2nsnc12 ; CC1(C)c2ccccc2N(c2ccccc2)c2ccc(-c3ccc(C=O)c4nsnc34)cc21 ; CC1(C)c2ccccc2N(c2ccccc2)c2ccc(-c3ccc(C=C(C#N)C#N)c4nsnc34)cc21]

After Compound 1-A (3.64 g, 8.8 mmol) and Compound 1-B (1.94 g, 8 mmol) were dissolved in tetrahydrofuran (THF)(200 ml) in a 2-neck round bottom flask and 2 M K₂CO₃ (100 ml) and a catalytic amount of Pd(PPh₃)₄ were put thereinto, the resulting mixture was refluxed for 5 hours, thereby obtaining Compound 1-C. Thereafter, the compound was purified through recrystallization, Compound 1-C (1.7 g, 3.8 mmol) was dissolved in tetrahydrofuran (100 ml), and malononitrile (500 mg) and a catalytic amount of piperidine were added thereto, thereby obtaining Compound 1 (1.13 g, 60%).

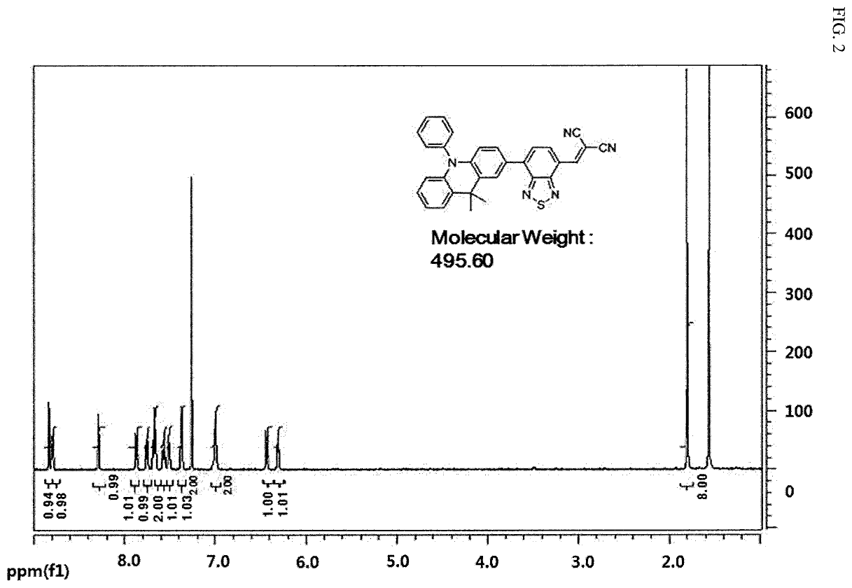

FIG. 2 is an FT-NMR graph of Compound 1.

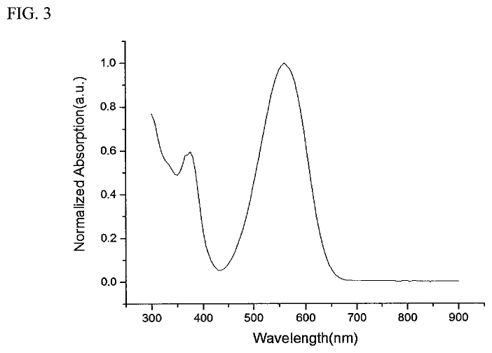

FIG. 3 is data obtained by measuring a UV-vis absorption spectrum in a solution state of Compound 1.

Specifically, FIG. 3 is data obtained by dissolving Compound 1 in toluene and measuring a UV-vis absorption spectrum thereof.

### Preparation Example 2. Preparation of Compound 2

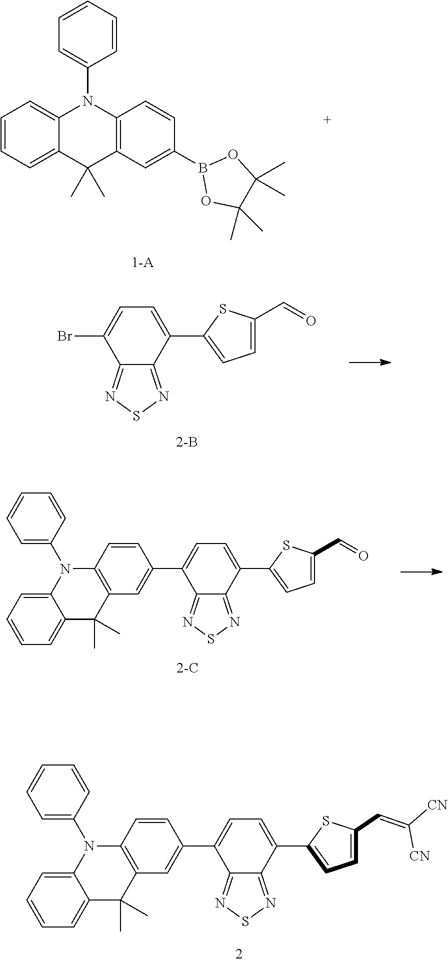

After Compound 1-A (3.64 g, 8.8 mmol) and Compound 2-B (2.6 g, 8 mmol) were dissolved in tetrahydrofuran (THF)(200 ml) in a 2-neck round bottom flask and 2 M K₂CO₃ (100 ml) and a catalytic amount of Pd(PPh₃)₄ were put thereinto, the resulting mixture was refluxed for 12 hours, thereby obtaining Compound 2-C (3.24 g) (yield=70%). Thereafter, the compound was purified through recrystallization, Compound 2-C (1.4 g, 2.64 mmol) was dissolved in tetrahydrofuran (100 ml), and malononitrile (700 mg) and a catalytic amount of piperidine were added thereto, thereby obtaining Compound 2 (1.2 g, 78%).

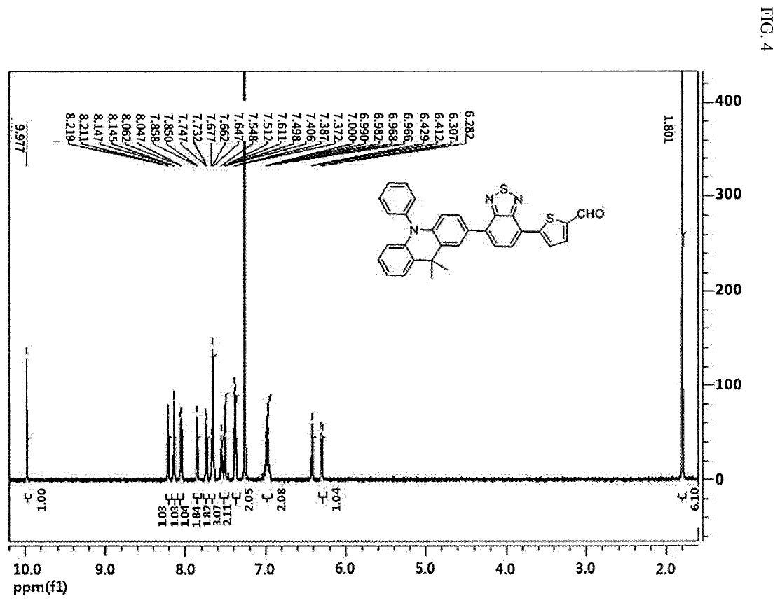

FIG. 4 is an FT-NMR graph of Compound 2-C, and FIG. 5 is an FT-NMR graph of Compound 2.

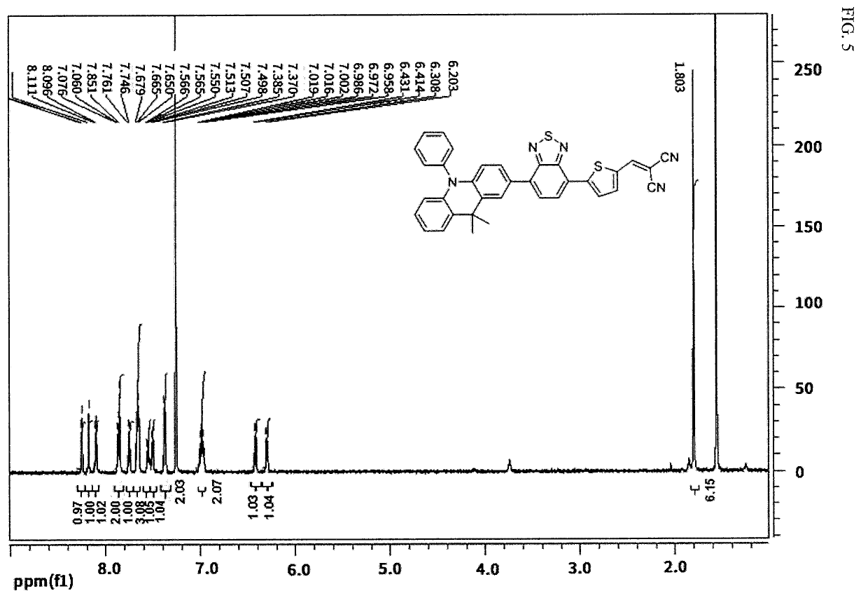

FIG. 6 is data obtained by measuring a UV-vis absorption spectrum in a solution state of Compound 2.

Specifically, FIG. 6 is data obtained by dissolving Compound 2 in toluene and measuring a UV-vis absorption spectrum thereof.

### Preparation Example 3. Preparation of Compound 3

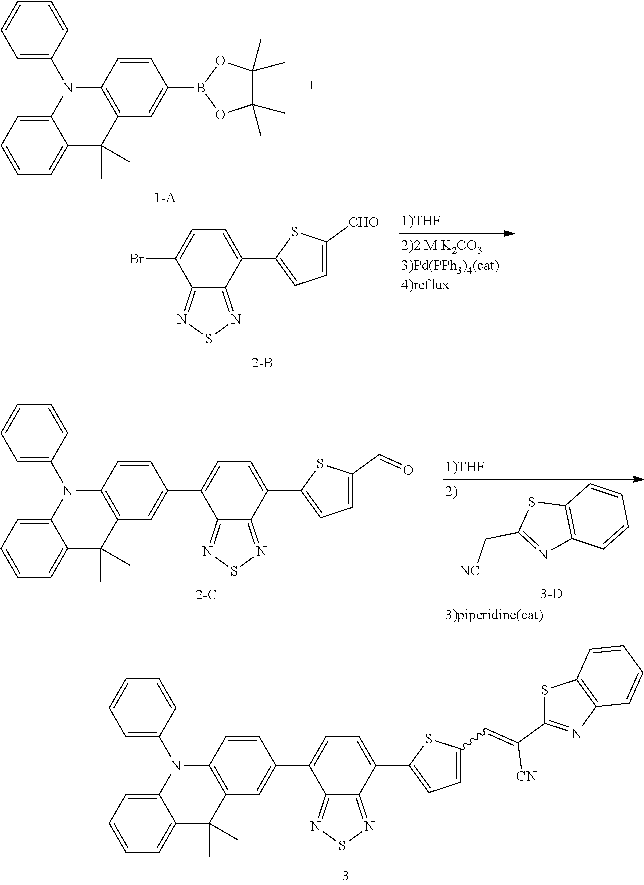 [SMILES: CC1(C)c2ccccc2N(c2ccccc2)c2ccc(B3OC(C)(C)C(C)(C)O3)cc21 ; O=Cc1ccc(-c2ccc(Br)c3nsnc23)s1 ; CC1(C)c2ccccc2N(c2ccccc2)c2ccc(-c3ccc(-c4ccc(C=O)s4)c4nsnc34)cc21 ; N#CCc1nc2ccccc2s1 ; CC1(C)c2ccccc2N(c2ccccc2)c2ccc(-c3ccc(-c4ccc(C=C(C#N)c5nc6ccccc6s5)s4)c4nsnc34)cc21]

After Compound 1-A (3.64 g, 8.8 mmol) and Compound 2-B (2.6 g, 8 mmol) were dissolved in tetrahydrofuran (THF)(200 ml) in a 2-neck round bottom flask and 2 M K₂CO₃ (100 ml) and a catalytic amount of Pd(PPh₃)₄ were put thereinto, the resulting mixture was refluxed for 12 hours, thereby obtaining Compound 2-C (3.57 g)(yield: 65%). Thereafter, the compound was purified through recrystallization, Compound 2-C (1.4 g, 2.64 mmol) was dissolved in tetrahydrofuran (100 ml), and Compound 3-D and a catalytic amount of piperidine were added thereto, thereby obtaining Compound 3.

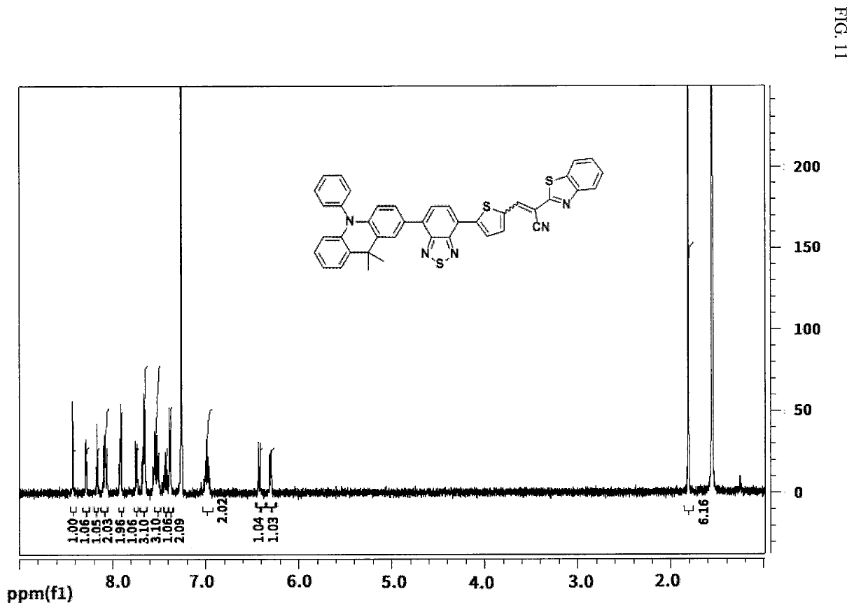

FIG. 11 is an FT-NMR graph of Compound 3.

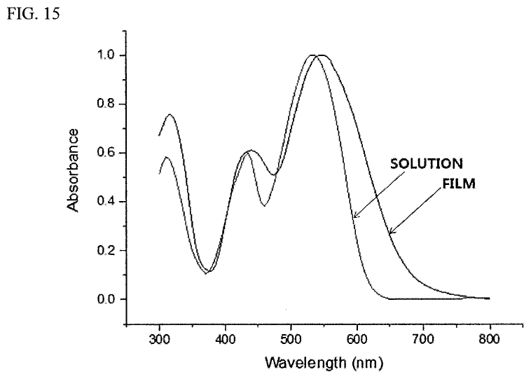

FIG. 15 is data obtained by measuring the UV-vis absorption spectra in the solution and film states of Compound 3. Specifically, FIG. 15 is data obtained by measuring the UV-vis absorption spectra of a sample of a solution obtained by dissolving Compound 3 in toluene and a film sample produced by dissolving Compound 3 in toluene.

In Compound 3, [symbol] means that isomers having a trans structure and a cis structure are mixed with each other.

### Preparation Example 4. Preparation of Compound 4

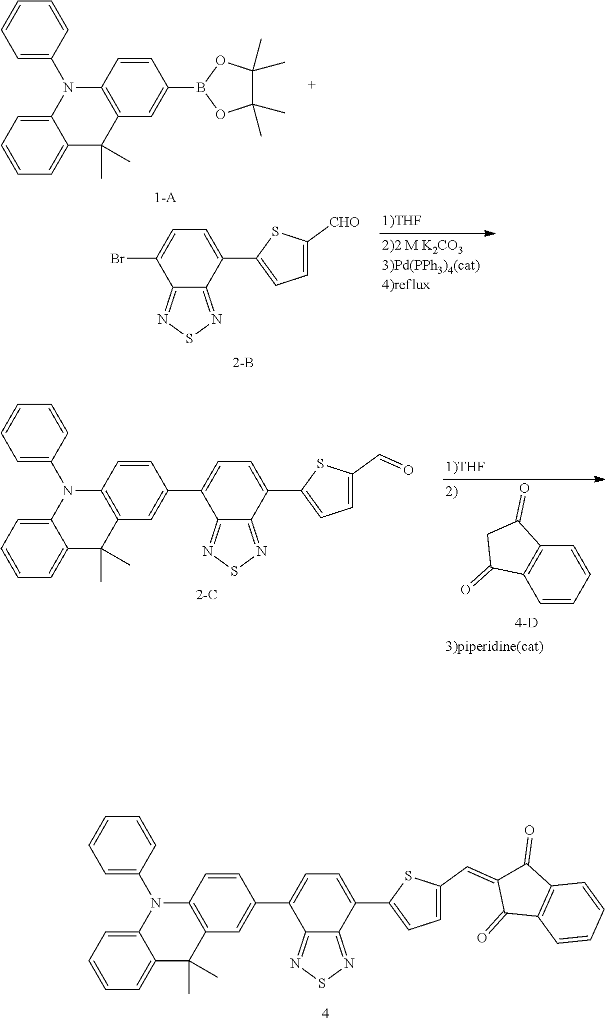 [SMILES: CC1(C)c2ccccc2N(c2ccccc2)c2ccc(B3OC(C)(C)C(C)(C)O3)cc21 ; O=Cc1ccc(-c2ccc(Br)c3nsnc23)s1 ; CC1(C)c2ccccc2N(c2ccccc2)c2ccc(-c3ccc(-c4ccc(C=O)s4)c4nsnc34)cc21 ; O=C1CC(=O)c2ccccc21 ; CC1(C)c2ccccc2N(c2ccccc2)c2ccc(-c3ccc(-c4ccc(C=C5C(=O)c6ccccc6C5=O)s4)c4nsnc34)cc21]

Compound 4 was obtained by performing the preparation in the same manner as in Preparation Example 3, except that Compound 4-D was used instead of Compound 3-D.

### Preparation Example 5. Preparation of Compound 5

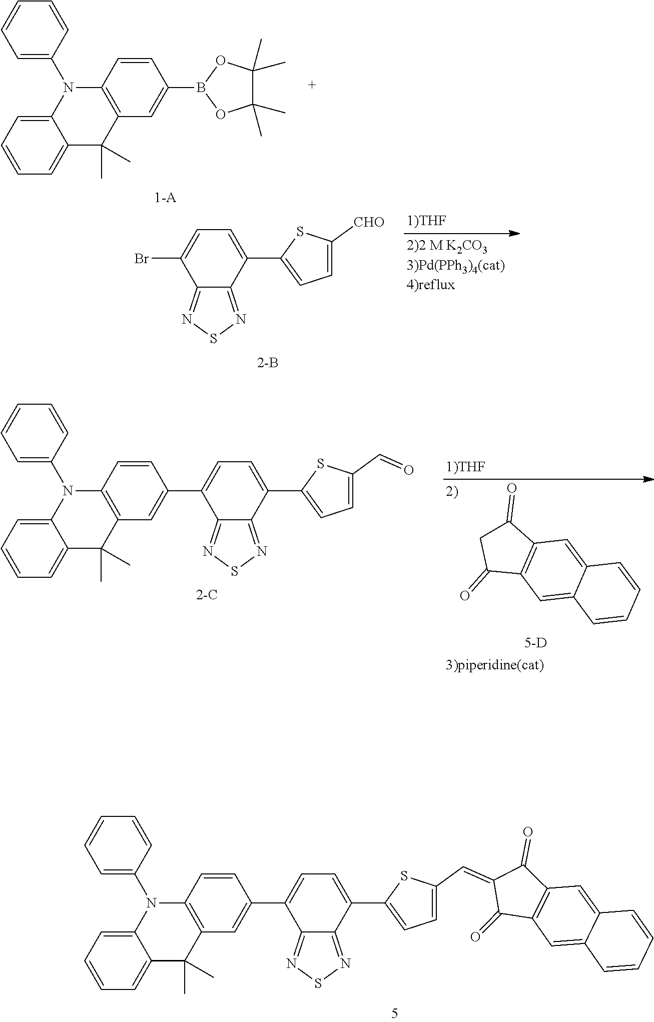 [SMILES: CC1(C)c2ccccc2N(c2ccccc2)c2ccc(B3OC(C)(C)C(C)(C)O3)cc21 ; O=Cc1ccc(-c2ccc(Br)c3nsnc23)s1 ; CC1(C)c2ccccc2N(c2ccccc2)c2ccc(-c3ccc(-c4ccc(C=O)s4)c4nsnc34)cc21 ; O=C1CC(=O)c2cc3ccccc3cc21 ; CC1(C)c2ccccc2N(c2ccccc2)c2ccc(-c3ccc(-c4ccc(C=C5C(=O)c6cc7ccccc7cc6C5=O)s4)c4nsnc34)cc21]

Compound 5 was obtained by performing the preparation in the same manner as in Preparation Example 3, except that Compound 5-D was used instead of Compound 3-D.

### Preparation Example 6. Preparation of Compound 6

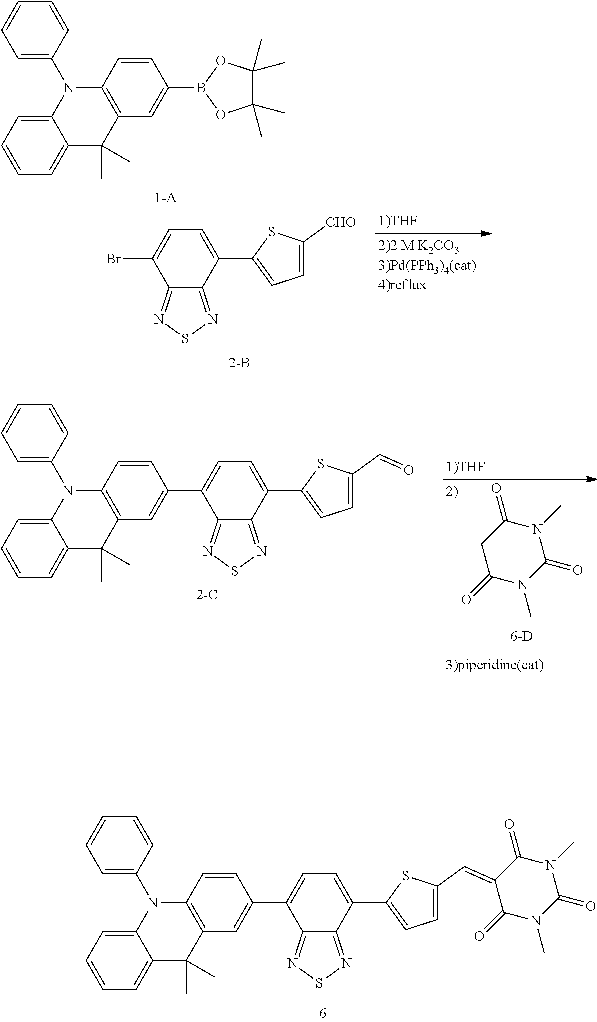 [SMILES: CC1(C)c2ccccc2N(c2ccccc2)c2ccc(B3OC(C)(C)C(C)(C)O3)cc21 ; O=Cc1ccc(-c2ccc(Br)c3nsnc23)s1 ; CC1(C)c2ccccc2N(c2ccccc2)c2ccc(-c3ccc(-c4ccc(C=O)s4)c4nsnc34)cc21 ; CN1C(=O)CC(=O)N(C)C1=O ; CN1C(=O)C(=Cc2ccc(-c3ccc(-c4ccc5c(c4)C(C)(C)c4ccccc4N5c4ccccc4)c4nsnc34)s2)C(=O)N(C)C1=O]

Compound 6 was obtained by performing the preparation in the same manner as in Preparation Example 3, except that Compound 6-D was used instead of Compound 3-D.

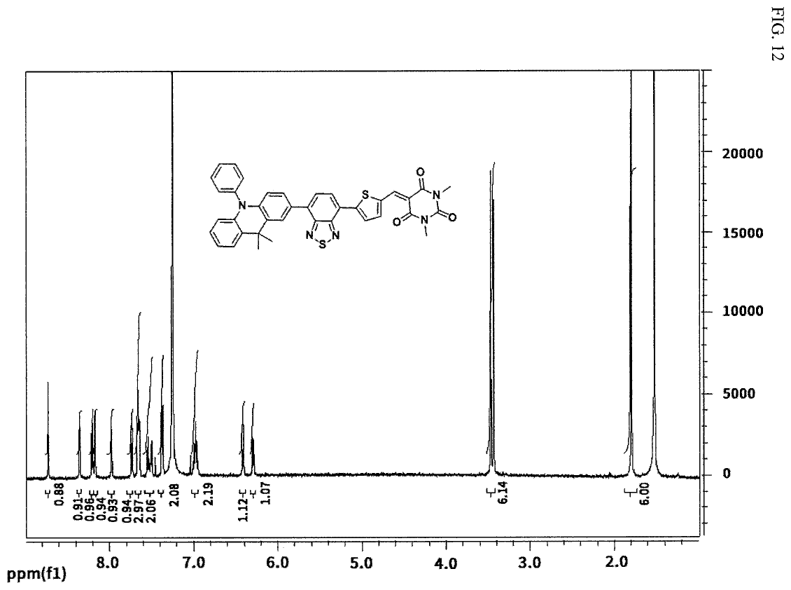

FIG. 12 is an FT-NMR graph of Compound 6.

### Preparation Example 7. Preparation of Compound 7

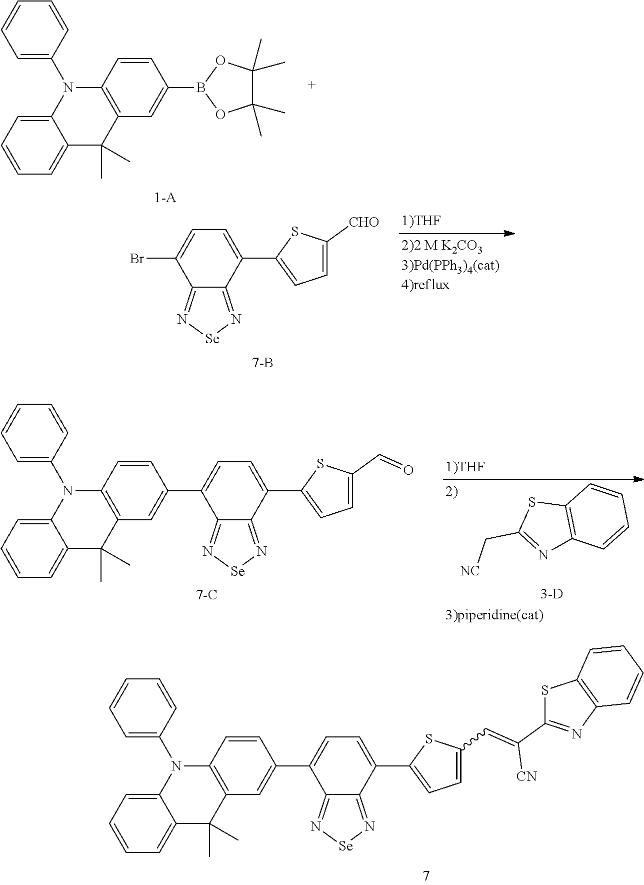 [SMILES: CC1(C)c2ccccc2N(c2ccccc2)c2ccc(B3OC(C)(C)C(C)(C)O3)cc21 ; O=Cc1ccc(-c2ccc(Br)c3n[se]nc23)s1 ; CC1(C)c2ccccc2N(c2ccccc2)c2ccc(-c3ccc(-c4ccc(C=O)s4)c4n[se]nc34)cc21 ; N#CCc1nc2ccccc2s1 ; CC1(C)c2ccccc2N(c2ccccc2)c2ccc(-c3ccc(-c4ccc(C=C(C#N)c5nc6ccccc6s5)s4)c4n[se]nc34)cc21]

After Compound 1-A (3.64 g, 8.8 mmol) and Compound 7-B (2.97 g, 8 mmol) were dissolved in tetrahydrofuran (THF)(200 ml) in a 2-neck round bottom flask and 2 M K₂CO₃ (100 ml) and a catalytic amount of Pd(PPh₃)₄ were put thereinto, the resulting mixture was refluxed for 12 hours, thereby obtaining Compound 7-C (3.81 g)(yield: 65%). Thereafter, the compound was purified through recrystallization, Compound 7-C (1.4 g, 2.64 mmol) was dissolved in tetrahydrofuran (100 ml), and Compound 3-D and a catalytic amount of piperidine were added thereto, thereby obtaining Compound 7.

In Compound 7, [symbol] means that isomers having a trans structure and a cis structure are mixed with each other.

### Preparation Example 8. Preparation of Compound 8

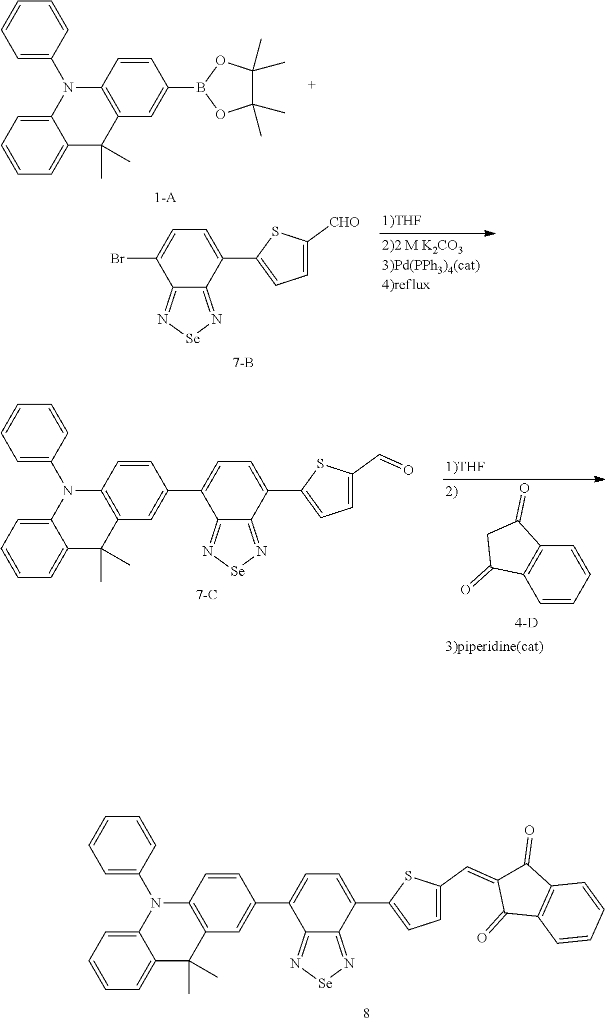 [SMILES: CC1(C)c2ccccc2N(c2ccccc2)c2ccc(B3OC(C)(C)C(C)(C)O3)cc21 ; O=Cc1ccc(-c2ccc(Br)c3n[se]nc23)s1 ; CC1(C)c2ccccc2N(c2ccccc2)c2ccc(-c3ccc(-c4ccc(C=O)s4)c4n[se]nc34)cc21 ; O=C1CC(=O)c2ccccc21 ; CC1(C)c2ccccc2N(c2ccccc2)c2ccc(-c3ccc(-c4ccc(C=C5C(=O)c6ccccc6C5=O)s4)c4n[se]nc34)cc21]

Compound 8 was obtained by performing the preparation in the same manner as in Preparation Example 7, except that Compound 4-D was used instead of Compound 3-D.

### Preparation Example 9. Preparation of Compound 9

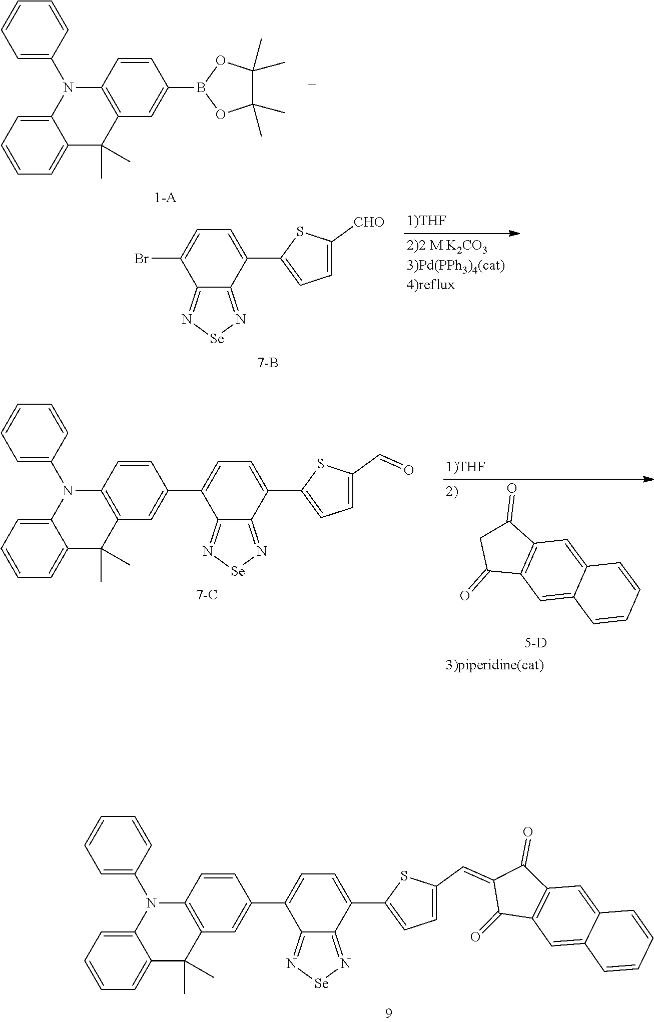 [SMILES: CC1(C)c2ccccc2N(c2ccccc2)c2ccc(B3OC(C)(C)C(C)(C)O3)cc21 ; O=Cc1ccc(-c2ccc(Br)c3n[se]nc23)s1 ; CC1(C)c2ccccc2N(c2ccccc2)c2ccc(-c3ccc(-c4ccc(C=O)s4)c4n[se]nc34)cc21 ; O=C1CC(=O)c2cc3ccccc3cc21 ; CC1(C)c2ccccc2N(c2ccccc2)c2ccc(-c3ccc(-c4ccc(C=C5C(=O)c6cc7ccccc7cc6C5=O)s4)c4n[se]nc34)cc21]

Compound 9 was obtained by performing the preparation in the same manner as in Preparation Example 7, except that Compound 5-D was used instead of Compound 3-D.

### Preparation Example 10. Preparation of Compound 10

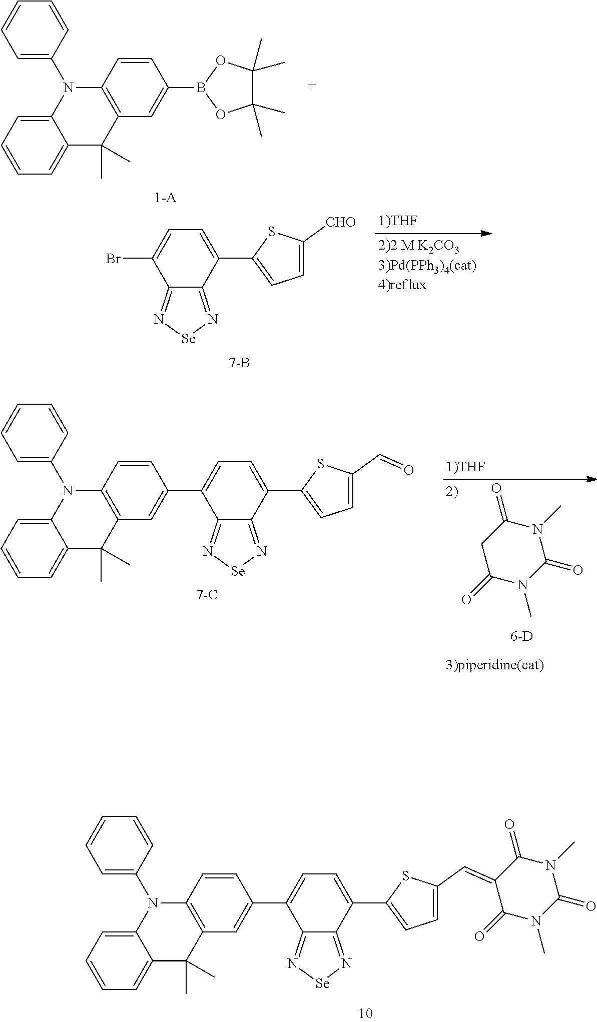 [SMILES: CC1(C)c2ccccc2N(c2ccccc2)c2ccc(B3OC(C)(C)C(C)(C)O3)cc21 ; O=Cc1ccc(-c2ccc(Br)c3n[se]nc23)s1 ; CC1(C)c2ccccc2N(c2ccccc2)c2ccc(-c3ccc(-c4ccc(C=O)s4)c4n[se]nc34)cc21 ; CN1C(=O)CC(=O)N(C)C1=O ; CN1C(=O)C(=Cc2ccc(-c3ccc(-c4ccc5c(c4)C(C)(C)c4ccccc4N5c4ccccc4)c4n[se]nc34)s2)C(=O)N(C)C1=O]

Compound 10 was obtained by performing the preparation in the same manner as in Preparation Example 7, except that Compound 6-D was used instead of Compound 3-D. Preparation Example 11. Preparation of Compound 11

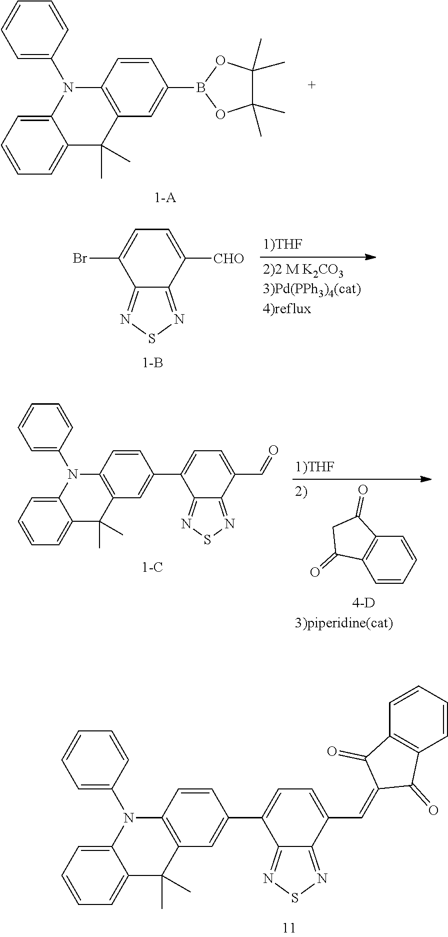 [SMILES: CC1(C)c2ccccc2N(c2ccccc2)c2ccc(B3OC(C)(C)C(C)(C)O3)cc21 ; O=Cc1ccc(Br)c2nsnc12 ; CC1(C)c2ccccc2N(c2ccccc2)c2ccc(-c3ccc(C=O)c4nsnc34)cc21 ; O=C1CC(=O)c2ccccc21 ; CC1(C)c2ccccc2N(c2ccccc2)c2ccc(-c3ccc(C=C4C(=O)c5ccccc5C4=O)c4nsnc34)cc21]

After Compound 1-A (3.64 g, 8.8 mmol) and Compound 1-B (1.94 g, 8 mmol) were dissolved in tetrahydrofuran (THF)(200 ml) in a 2-neck round bottom flask and 2 M K₂CO₃ (100 ml) and a catalytic amount of Pd(PPh₃)₄ were put thereinto, the resulting mixture was refluxed for 12 hours, thereby obtaining Compound 1-C. Thereafter, the compound was purified through recrystallization, Compound 1-C (1.4 g, 3.12 mmol) was dissolved in tetrahydrofuran (100 ml), and Compound 4-D and a catalytic amount of piperidine were added thereto, thereby obtaining Compound 11.

### Preparation Example 12. Preparation of Compound 12

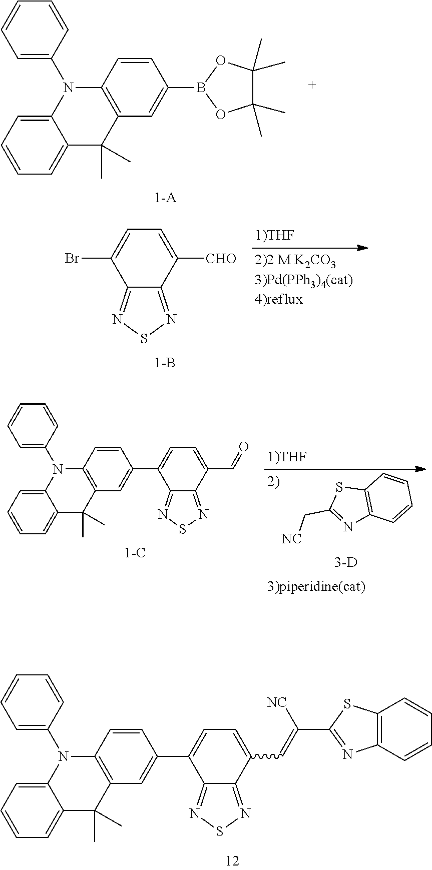 [SMILES: CC1(C)c2ccccc2N(c2ccccc2)c2ccc(B3OC(C)(C)C(C)(C)O3)cc21 ; O=Cc1ccc(Br)c2nsnc12 ; CC1(C)c2ccccc2N(c2ccccc2)c2ccc(-c3ccc(C=O)c4nsnc34)cc21 ; N#CCc1nc2ccccc2s1 ; CC1(C)c2ccccc2N(c2ccccc2)c2ccc(-c3ccc(C=C(C#N)c4nc5ccccc5s4)c4nsnc34)cc21]

Compound 12 was obtained by performing the preparation in the same manner as in Preparation Example 11, except that Compound 3-D was used instead of Compound 4-D.

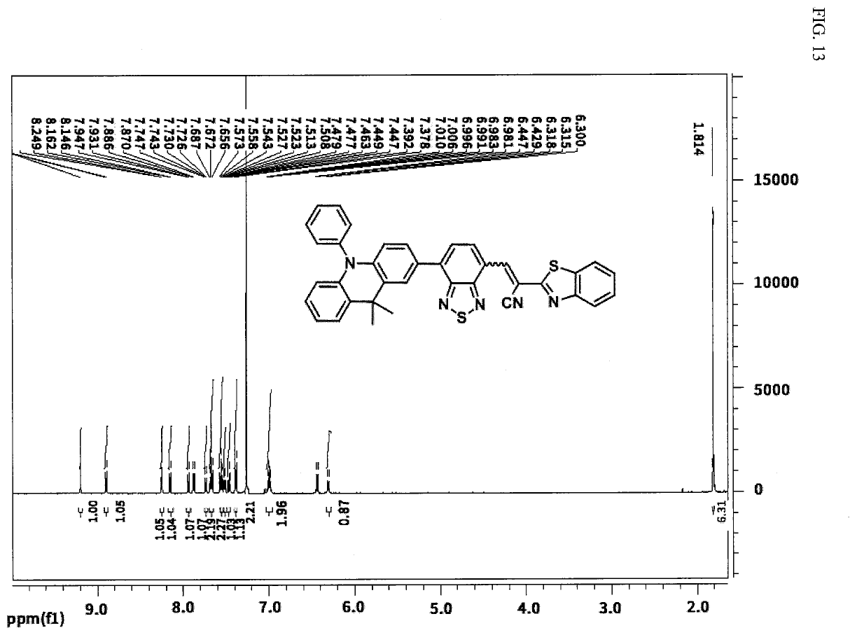

FIG. 13 is an FT-NMR graph of Compound 12.

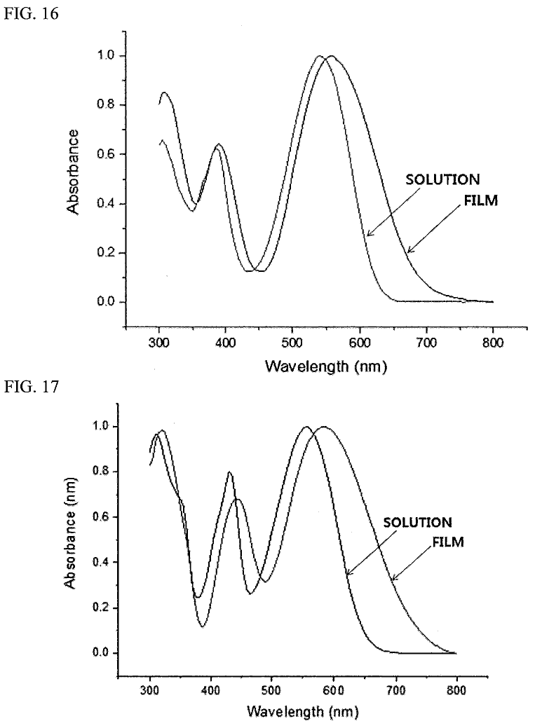

FIG. 16 is data obtained by measuring the UV-vis absorption spectra in the solution and film states of Compound 12. Specifically, FIG. 16 is data obtained by measuring the UV-vis absorption spectra of a sample of a solution obtained by dissolving Compound 12 in toluene and a film sample produced by dissolving Compound 12 in toluene.

In Compound 12, [symbol] means that isomers having a trans structure and a cis structure are mixed with each other.

### Preparation Example 13. Preparation of Compound 13

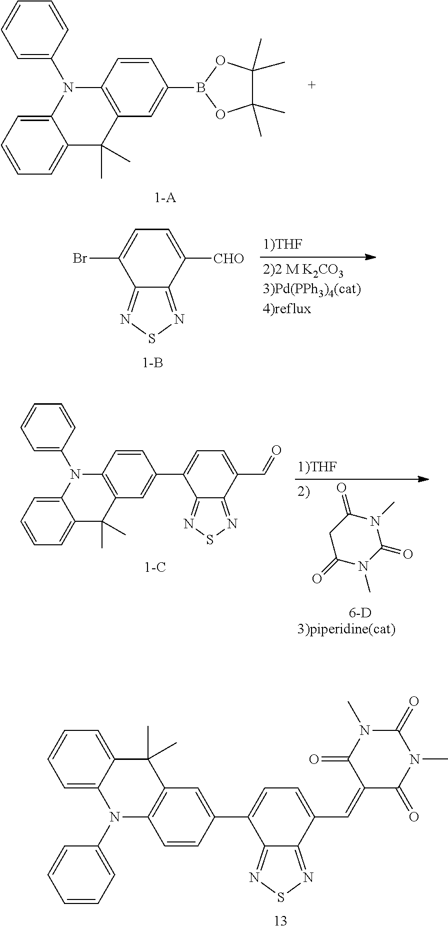 [SMILES: CC1(C)c2ccccc2N(c2ccccc2)c2ccc(B3OC(C)(C)C(C)(C)O3)cc21 ; O=Cc1ccc(Br)c2nsnc12 ; CC1(C)c2ccccc2N(c2ccccc2)c2ccc(-c3ccc(C=O)c4nsnc34)cc21 ; CN1C(=O)CC(=O)N(C)C1=O ; CN1C(=O)C(=Cc2ccc(-c3ccc4c(c3)C(C)(C)c3ccccc3N4c3ccccc3)c3nsnc23)C(=O)N(C)C1=O]

Compound 13 was obtained by performing the preparation in the same manner as in Preparation Example 11, except that Compound 6-D was used instead of Compound 4-D.

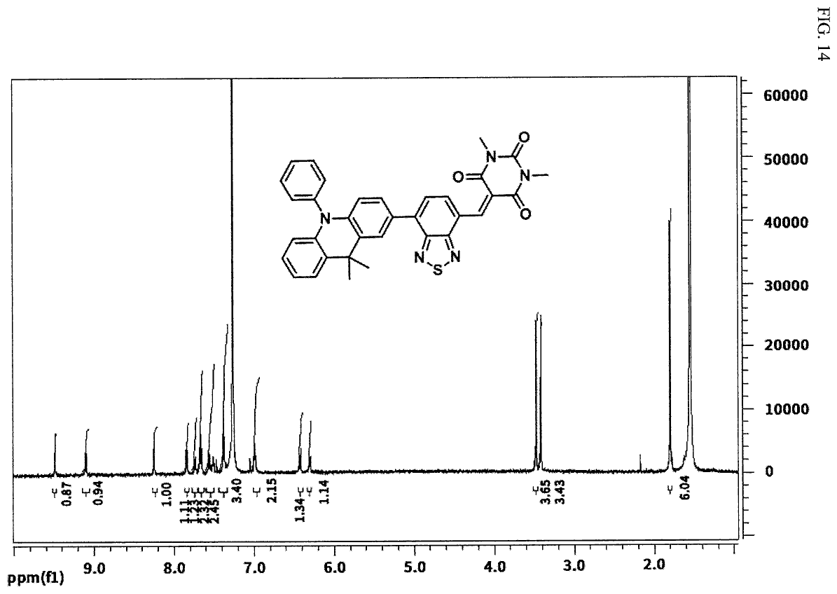

FIG. 14 is an FT-NMR graph of Compound 13.

### Preparation Example 14. Preparation of Compound 14

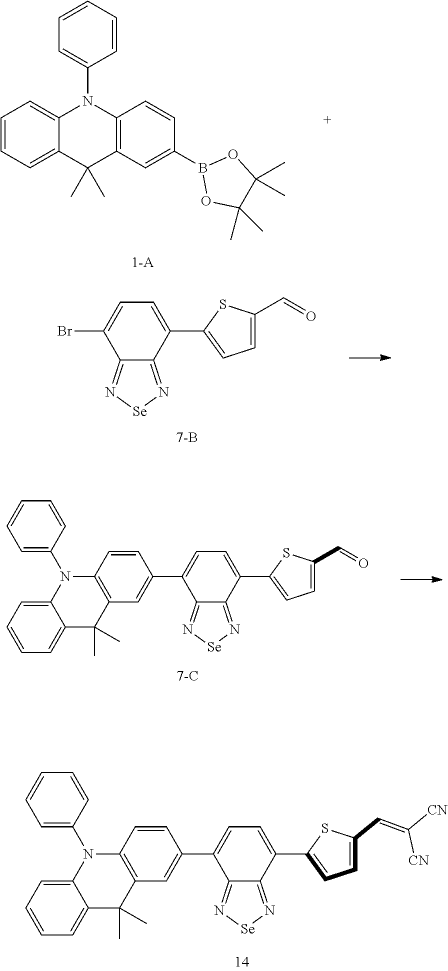

Compound 14 was obtained by performing the preparation in the same manner as in Preparation Example 2, except that Compound 7-B was used instead of Compound 2-B.

FIG. 17 is data obtained by measuring the UV-vis absorption spectra in the solution and film states of Compound 14. Specifically, FIG. 17 is data obtained by measuring the UV-vis absorption spectra of a sample of a solution obtained by dissolving Compound 14 in toluene and a film sample produced by dissolving Compound 14 in toluene.

### Preparation Example 15. Preparation of Compound 15

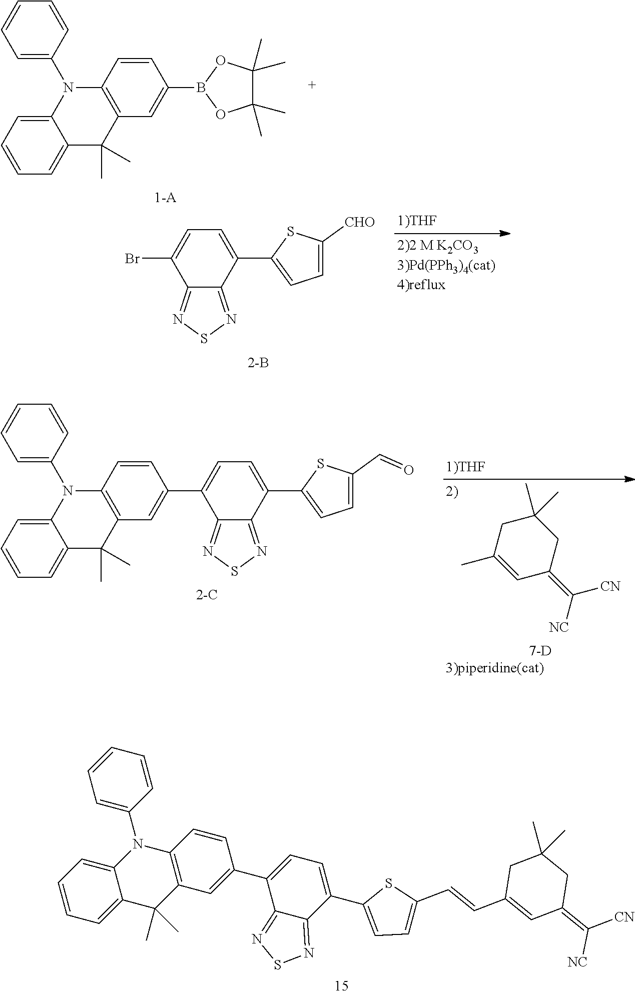 [SMILES: CC1(C)c2ccccc2N(c2ccccc2)c2ccc(B3OC(C)(C)C(C)(C)O3)cc21 ; O=Cc1ccc(-c2ccc(Br)c3nsnc23)s1 ; CC1(C)c2ccccc2N(c2ccccc2)c2ccc(-c3ccc(-c4ccc(C=O)s4)c4nsnc34)cc21 ; CC1=CC(=C(C#N)C#N)CC(C)(C)C1 ; CC1(C)CC(/C=C/c2ccc(-c3ccc(-c4ccc5c(c4)C(C)(C)c4ccccc4N5c4ccccc4)c4nsnc34)s2)=CC(=C(C#N)C#N)C1]

Compound 15 was obtained by performing the preparation in the same manner as in Preparation Example 3, except that Compound 7-D was used instead of Compound 3-D.

Manufacture of Organic Photoelectric Device

### Example 1-1

An organic photoelectric device was manufactured to have a normal structure of ITO/MoO₃/a photoactive layer/BCP/Al. For the ITO, an anode was formed of an organic substrate (11.5 Ω/□, 1.1 t) coated with 0.2 cm×0.2 cm of a pinwheel pattern, and ultrasonically washed by using distilled water, acetone, and 2-propanol, and a molybdenum oxide (MoO₃) thin film as a hole transport layer was stacked to a thickness of 30 nm at a rate of 1.0 Å/s thereon. Next, a photoactive layer having a thickness of 100 nm was formed by co-depositing the Compound 1 (a p-type organic material layer) and C₆₀ (an n-type organic material layer) according to Preparation Example 1 at a thickness ratio of 1:1 on the molybdenum oxide (MoO₃) thin film. Next, bathocuproine (BCP) as an electron transport layer was stacked to a thickness of 8 nm at a rate of 1.0 Å/s on the photoactive layer, and a cathode having a thickness of 100 nm was formed thereon by stacking aluminum (Al) by sputtering, thereby manufacturing an organic photoelectric device.

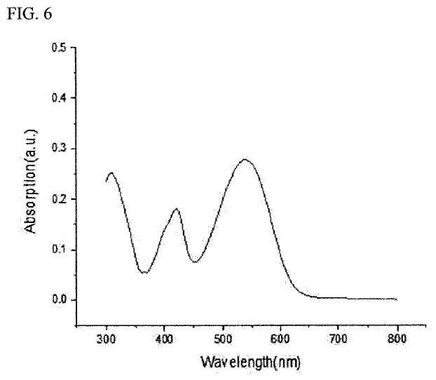

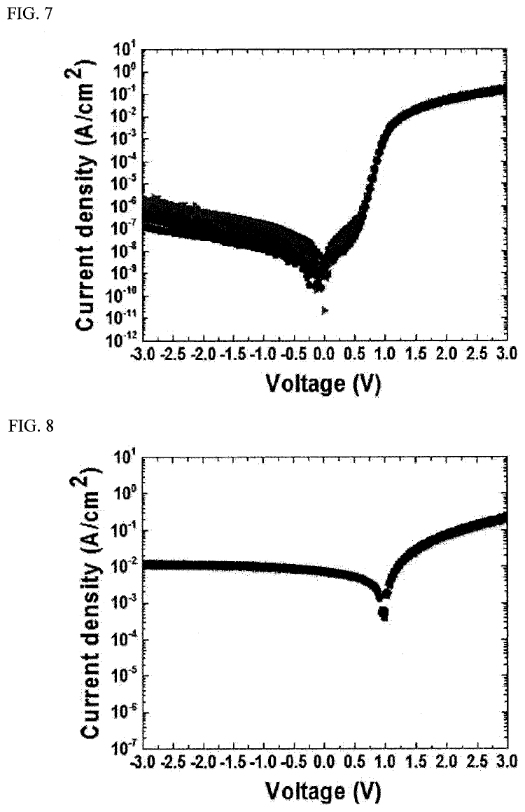

FIG. 7 is a current density graph of the organic photoelectric device manufactured in Example 1-1 over voltage at dark current, and FIG. 8 is a current density graph of the organic photoelectric device manufactured in Example 1-1 over voltage at photoelectric current. Specifically, according to FIGS. 7 and 8, it can be seen that the current value of the organic photoelectric device manufactured in Example 1-1 is constant, and each layer of the organic photoelectric device is stably deposited.

### Example 1-2

An organic photoelectric device was manufactured to have an inverted structure of ITO/BCP/a photoactive layer/MoO₃/Al. For the ITO, a cathode was formed of an organic substrate (11.5 Ω/□, 1.1 t) coated with 0.2 cm×0.2 cm of a pinwheel pattern, and ultrasonically washed by using distilled water, acetone, and 2-propanol, and bathocuproine (BCP) as an electron transport layer was stacked to a thickness of 8 nm at a rate of 1.0 Å/s thereon, and a photoactive layer having a thickness of 100 nm was formed thereon by co-depositing Compound 1 (a p-type organic material layer) and C₆₀ (an n-type organic material layer) according to Preparation Example 1 at a thickness ratio of 1:1. A molybdenum oxide (MoO₃) thin film as a hole transport layer was stacked to a thickness of 30 nm at a rate of 1.0 Å/s on the photoactive layer. An anode having a thickness of 100 nm was formed by stacking Aluminum (Al) on the hole transport layer by sputtering, thereby manufacturing an organic photoelectric device.

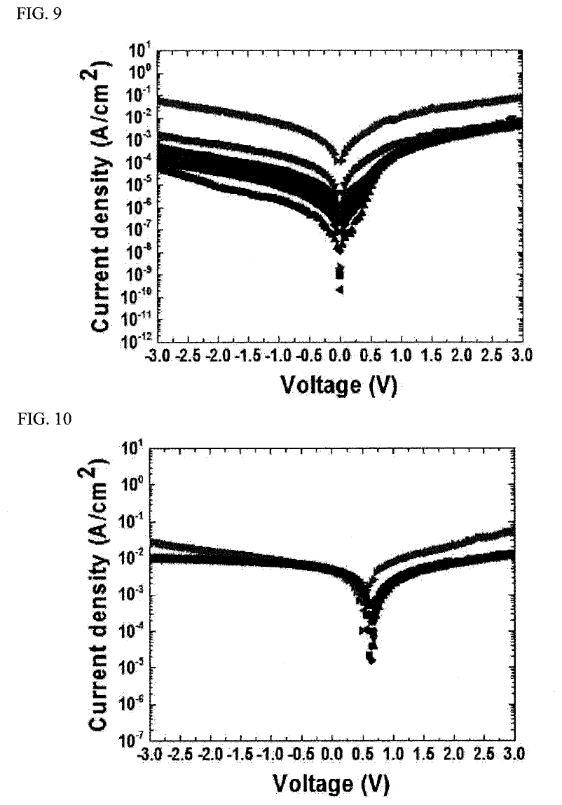

FIG. 9 is a current density graph of the organic photoelectric device manufactured in Example 1-2 over voltage at dark current, and FIG. 10 is a current density graph of the organic photoelectric device manufactured in Example 1-2 over voltage at photoelectric current.

The photoelectric conversion characteristics of the organic photoelectric devices manufactured in Examples 1-1 and 1-2 were measured under the conditions of 0 mW/cm² (−1 V or −3 V) and 100 mW/cm² (AM 1.5), and the results thereof are shown in the following Table 1.

TABLE 1

| | J_{dark} at −1 V | J_{dark} at −3 V | J_{SC} | V_{OC} | PCE |
|---|---|---|---|---|---|
| | (nA/cm²) | (nA/cm²) | (mA/cm²) | (V) | (%) |
| Example 1-1 | 12.69 | 124.35 | 7.35 | 0.98 | 2.799 |
| Example 1-2 | 2.14 × 10³ | 5.97 × 10⁴ | 5.10 | 0.67 | 1.065 |

In Table 1, J_{dark}, V_{oc}, J_{sc}, and PCE(η) mean a dark current, an open-circuit voltage, a short-circuit current, and an energy conversion efficiency, respectively. The open-circuit voltage and the short-circuit current are an X axis intercept and a Y axis intercept, respectively, in the fourth quadrant of the voltage-current density curve, and as the two values are increased, the PCE is increased. According to the results in Table 1, it can be seen that the organic photoelectric devices in Examples 1-1 and 1-2 have excellent photoelectric conversion efficiencies. The external quantum efficiency (EQE) of each of the organic photoelectric devices manufactured in Examples 1-1 and 1-2 was evaluated over wavelength and voltage.

For the external quantum efficiency, the IPCE measurement is performed by using a (PV measurement, USA) facility. First, the facility was calibrated by using an Si photodiode (manufactured by Hamamatsu Photonics K.K., Japan), and then the organic photoelectric devices according to Examples 1-1 and 1-2 were mounted in the facility, the external quantum efficiency was measured in a region with a voltage of −3 V and 0 V and a wavelength range of 300 to 800 nm, and the results thereof are shown in the following Table

TABLE 2

| | Maximum external quantum efficiency (%) | | | | | |
|---|---|---|---|---|---|---|
| | at 0 V | λ(nm) | at −1 V | λ(nm) | at −3 V | λ(nm) |
| Example 1-1 | 57.78 | 570 | 64.88 | 580 | 70.81 | 570 |
| Example 1-2 | 41.62 | 390 | 54.67 | 400 | 64.05 | 400 |

According to the results in Table 2, since the organic photoelectric devices in Examples 1-1 and 1-2 include the heterocyclic compound represented by Formula 1 as the photoactive layer, the organic photoelectric devices include acridine which serves as an electron donor, so that an intermolecular stacking is effectively achieved by adjusting the intermolecular planarity. Further, in the heterocyclic compound represented by Formula 1, the band gap is reduced by inserting a benzothiadiazole group which serves as an electron acceptor into a linking group. Accordingly, the organic photoelectric devices in Examples 1-1 and 1-2 have high external quantum efficiency over wavelength and voltage.

### Example 2-1

An organic photoelectric device was manufactured in the same manner as in Example 1-1, except that Compound 2 was used instead of Compound 1 as the photoactive layer.

### Example 2-2

An organic photoelectric device was manufactured in the same manner as in Example 1-2, except that Compound 2 was used instead of Compound 1 as the photoactive layer.

The external quantum efficiency (EQE) and short-circuit current of each of the organic photoelectric devices manufactured in Examples 2-1 and 2-2 were evaluated over wavelength and voltage.

For the external quantum efficiency, the IPCE measurement is performed by using a (PV measurement, USA) facility. First, the facility was calibrated by using an Si photodiode (manufactured by Hamamatsu Photonics K.K., Japan), and then the organic photoelectric devices according to Examples 2-1 and 2-2 were mounted in the facility, the external quantum efficiency was measured in a region with a voltage of −3 V and 0 V and a wavelength range of 300 to 800 nm, the short-circuit current was measured under 0 lux and 12355 lux (−3 V) conditions, and the results thereof are shown in the following Table 3.

TABLE 3

| | Maximum external | | | | | |
|---|---|---|---|---|---|---|
| | quantum efficiency | | | | | |
| | (%) | | | | Short-circuit current | |
| | at | | at | | (A/cm²) at −3 V | |
| | 0 V | λ(nm) | −3 V | λ(nm) | 0 lux | 12355 lux |
| Example | 59.1 | 550 | 71.2 | 550 | 1.11 × 10⁻⁷ | 1.25 × 10⁻³ |
| 2-1 | 60.0 | 550 | 72.0 | 550 | 1.12 × 10⁻⁷ | 1.24 × 10⁻³ |
| Example | 49.9 | 400 | 62.9 | 400 | 1.57 × 10⁻⁶ | 1.19 × 10⁻³ |
| 2-2 | 49.3 | 390 | 62.7 | 430 | 6.50 × 10⁻⁶ | 1.14 × 10⁻³ |

According to the results in Table 3, since the organic photoelectric devices in Examples 2-1 and 2-2 include the heterocyclic compound represented by Formula 1 as the photoactive layer, the organic photoelectric devices include acridine which serves as an electron donor, so that an intermolecular stacking is effectively achieved by adjusting the intermolecular planarity. Further, in the heterocyclic compound represented by Formula 1, the band gap is reduced by inserting a thiophene group which serves as an electron donor and a benzothiadiazole group which serves as an electron acceptor into a linking group. Accordingly, it can be seen that the organic photoelectric devices in Examples 2-1 and 2-2 have high external quantum efficiencies over wavelength and voltage, and have excellent efficiencies.

### Example 3-1

An organic photoelectric device was manufactured in the same manner as in Example 1-1, except that Compound 3 was used instead of Compound 1 as the photoactive layer.

### Example 3-2

An organic photoelectric device was manufactured in the same manner as in Example 1-2, except that Compound 3 was used instead of Compound 1 as the photoactive layer.

For the external quantum efficiency, the IPCE measurement is performed by using a (PV measurement, USA) facility. First, the facility was calibrated by using an Si photodiode (manufactured by Hamamatsu Photonics K.K., Japan), and then the organic photoelectric devices according to Examples 3-1 and 3-2 were mounted in the facility, the external quantum efficiency was measured in a region with a voltage of −3 V and 0 V and a wavelength range of 300 to 800 nm, values for the dark current, photoelectric current, and external quantum efficiency of each of the organic photoelectric devices in Examples 3-1 and 3-2 were measured under the −3 V condition, and the results thereof are shown in the following Table 4.

TABLE 4

| | J_{dark} at −1 V | Jₚₕₒₜₒ at −3 V | EQE at −3 V |
|---|---|---|---|
| | (A/cm²) | (A/cm²) | (%) |
| Example 3-1 | 9.6 × 10⁻⁹ | 1.22 × 10⁻³ | 66.4 (at 550 nm) |
| Example 3-2 | 1.24 × 10⁻⁷ | 1.10 × 10⁻³ | 59.6 (at 560 nm) |

In Table 4, J_{dark}, Jₚₕₒₜₒ, and EQE mean the dark current, the photoelectric current, and the external quantum efficiency, respectively, the organic photoelectric device in Example 3-1 exhibits a maximum external quantum efficiency at 550 nm, and the organic photoelectric device in Example 3-2 exhibits a maximum external quantum efficiency at 560 nm. According to the results in Table 4, since the organic photoelectric devices in Examples 3-1 and 3-2 include the heterocyclic compound represented by Formula 1 as the photoactive layer, the organic photoelectric devices include acridine which serves as an electron donor, so that an intermolecular stacking is effectively achieved by adjusting the intermolecular planarity. Further, in the heterocyclic compound represented by Formula 1, the band gap is reduced by inserting a thiophene group which serves as an electron donor, a benzothiadiazole group which serves as an electron acceptor, and a benzothiazole group which serves as an end group into a linking group. Accordingly, the organic photoelectric devices in Examples 3-1 and 3-2 have constant dark current and photoelectric current over voltage and high external quantum efficiency over wavelength and voltage.

### Example 4-1

An organic photoelectric device was manufactured in the same manner as in Example 1-1, except that Compound 12 was used instead of Compound 1 as the photoactive layer.

### Example 4-2

An organic photoelectric device was manufactured in the same manner as in Example 1-2, except that Compound 12 was used instead of Compound 1 as the photoactive layer.

For the external quantum efficiency, the IPCE measurement is performed by using a (PV measurement, USA) facility. First, the facility was calibrated by using an Si photodiode (manufactured by Hamamatsu Photonics K.K., Japan), and then the organic photoelectric devices according to Examples 4-1 and 4-2 were mounted in the facility, the external quantum efficiency was measured in a region with a voltage of −3 V and 0 V and a wavelength range of 300 to 700 nm, values for the dark current, photoelectric current, and external quantum efficiency of each of the organic photoelectric devices in Examples 4-1 and 4-2 were measured under the −3 V condition, and the results thereof are shown in the following Table 5.

TABLE 5

| | J_{dark} at −1 V | Jₚₕₒₜₒ at −3 V | EQE at −3 V |
|---|---|---|---|
| | (A/cm²) | (A/cm²) | (%) |
| Example 4-1 | 7.71 × 10⁻⁹ | 5.55 × 10⁻³ | 51.8 (at 550 nm) |
| Example 4-2 | 9.29 × 10⁻⁶ | 6.89 × 10⁻³ | 47.0 (at 400 nm) |

In Table 5, J_{dark}, Jₚₕₒₜₒ, and EQE mean the dark current, the photoelectric current, and the external quantum efficiency, respectively, the organic photoelectric device in Example 4-1 exhibits a maximum external quantum efficiency at 550 nm, and the organic photoelectric device in Example 4-2 exhibits a maximum external quantum efficiency at 400 nm. According to the results in Table 5, since the organic photoelectric devices in Examples 4-1 and 4-2 include the heterocyclic compound represented by Formula 1 as the photoactive layer, the organic photoelectric devices include acridine which serves as an electron donor, so that an intermolecular stacking is effectively achieved by adjusting the intermolecular planarity. Further, in the heterocyclic compound represented by Formula 1, the band gap is reduced by inserting a benzothiadiazole group which serves as an electron acceptor into a linking group and adding a benzothiazole group to an end group. Accordingly, the organic photoelectric devices in Examples 4-1 and 4-2 have constant dark current and photoelectric current over voltage and high external quantum efficiency over wavelength and voltage.

### Example 5-1

An organic photoelectric device was manufactured in the same manner as in Example 1-1, except that Compound 14 was used instead of Compound 1 as the photoactive layer.

### Example 5-2

An organic photoelectric device was manufactured in the same manner as in Example 1-2, except that Compound 14 was used instead of Compound 1 as the photoactive layer.

For the external quantum efficiency, the IPCE measurement is performed by using a (PV measurement, USA) facility. First, the facility was calibrated by using an Si photodiode (manufactured by Hamamatsu Photonics K.K., Japan), and then the organic photoelectric devices according to Examples 5-1 and 5-2 were mounted in the facility, the external quantum efficiency was measured in a region with a voltage of −3 V and 0 V and a wavelength range of 300 to 800 nm, values for the dark current, photoelectric current, and external quantum efficiency of each of the organic photoelectric devices in Examples 5-1 and 5-2 were measured under the −3 V condition, and the results thereof are shown in the following Table 6.

TABLE 6

| | J_{dark} at −1 V | Jₚₕₒₜₒ at −3 V | EQE at −3 V |
|---|---|---|---|
| | (A/cm²) | (A/cm²) | (%) |
| Example 5-1 | 1.15 × 10⁻⁸ | 1.31 × 10⁻³ | 68.6 (at 570 nm) |
| Example 5-2 | 7.53 × 10⁻⁸ | 1.13 × 10⁻³ | 59.3 (at 410 nm) |

In Table 6, J_{dark}, Jₚₕₒₜₒ, and EQE mean the dark current, the photoelectric current, and the external quantum efficiency, respectively, the organic photoelectric device in Example 5-1 exhibits a maximum external quantum efficiency at 570 nm, and the organic photoelectric device in Example 5-2 exhibits a maximum external quantum efficiency at 410 nm. According to the results in Table 6, since the organic photoelectric devices in Examples 5-1 and 5-2 include the heterocyclic compound represented by Formula 1 as the photoactive layer, the organic photoelectric devices include acridine which serves as an electron donor, so that an intermolecular stacking is effectively achieved by adjusting the intermolecular planarity. Further, in the heterocyclic compound represented by Formula 1, the band gap is reduced by inserting a thiophene group which serves as an electron donor and a benzoselenadiazole group which serves as an electron acceptor into a linking group. Accordingly, the organic photoelectric devices in Examples 5-1 and 5-2 have constant dark current and photoelectric current over voltage and high external quantum efficiency over wavelength and voltage.

### Example 6-1

An organic photoelectric device was manufactured in the same manner as in Example 1-1, except that Compound 15 was used instead of Compound 1 as the photoactive layer.

### Example 6-2

An organic photoelectric device was manufactured in the same manner as in Example 1-2, except that Compound 15 was used instead of Compound 1 as the photoactive layer.

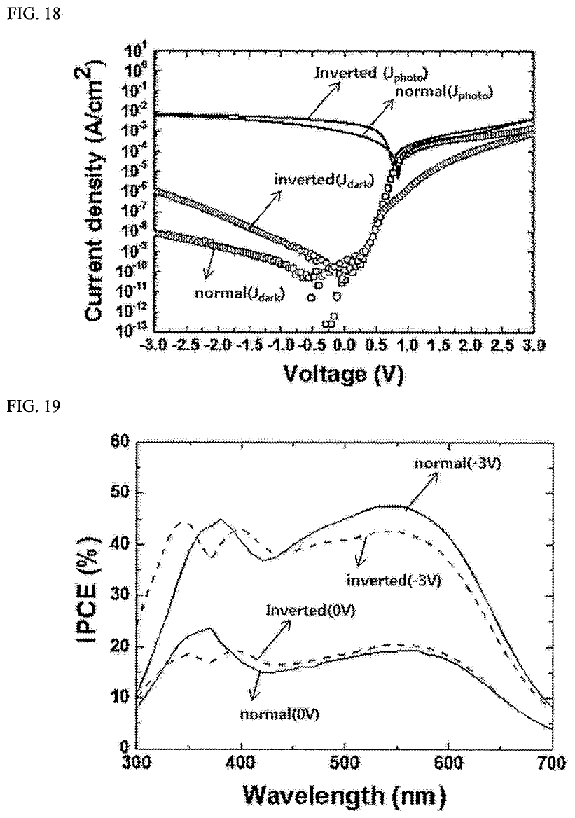

FIG. 18 is a current density graph of the organic photoelectric devices manufactured in Examples 6-1 and 6-2 over voltage at photoelectric current and dark current.

Further, FIG. 19 is a graph illustrating the external quantum efficiencies of the organic photoelectric devices manufactured in Examples 6-1 and 6-2 over wavelength and voltage.

For the external quantum efficiency of FIG. 19, the IPCE measurement is performed by using a (PV measurement, USA) facility. First, the facility was calibrated by using an Si photodiode (manufactured by Hamamatsu Photonics K.K., Japan), and then the organic photoelectric devices according to Examples 6-1 and 6-2 were mounted in the facility, and the external quantum efficiency was measured in a region with a voltage of −3 V and 0 V and a wavelength range of 300 to 700 nm.

Among the data in FIGS. 18 and 19, values for the dark current, photoelectric current, and external quantum efficiency of each of the organic photoelectric devices in Examples 6-1 and 6-2 were measured under the −3 V condition, and the results thereof are shown in the following Table 7.

TABLE 7

| | J_{dark} at −1 V | Jₚₕₒₜₒ at −3 V | EQE at −3 V |
|---|---|---|---|
| | (A/cm²) | (A/cm²) | (%) |
| Example 6-1 | 9.14 × 10⁻⁹ | 6.38 × 10⁻³ | 47.6 (at 540 nm) |
| Example 6-2 | 1.09 × 10⁻⁶ | 7.13 × 10⁻³ | 42.7 (at 550 nm) |

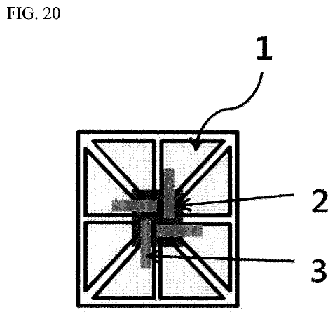

In Table 7 and FIGS. 18 and 19, J_{dark}, Jₚₕₒₜₒ, and EQE mean the dark current, the photoelectric current, and the external quantum efficiency, respectively, the organic photoelectric device in Example 6-1 exhibits a maximum external quantum efficiency at 540 nm, and the organic photoelectric device in Example 6-2 exhibits a maximum external quantum efficiency at 550 nm. According to the results in Table 7 and FIGS. 18 and 19, since the organic photoelectric devices in Examples 6-1 and 6-2 include the heterocyclic compound represented by Formula 1 as the photoactive layer, the organic photoelectric devices include acridine which serves as an electron donor, so that an intermolecular stacking is effectively achieved by adjusting the intermolecular planarity. Further, in the heterocyclic compound represented by Formula 1, the band gap is reduced by inserting a thiophene group which serves as an electron donor and a benzothiadiazole group which serves as an electron acceptor into a linking group. Therefore, the organic photoelectric devices in Examples 6-1 and 6-2 have constant dark current and photoelectric current over voltage and high external quantum efficiency over wavelength and voltage. FIG. 20 is a top view of the organic photoelectric devices manufactured in Examples 1-1, 1-2, 2-1, 2-2, 3-1, 3-2, 4-1, 4-2, 5-1, 5-2, 6-1, and 6-2, and in FIG. 20, (1), (2), and (3) mean an anode (a cathode), an organic material layer, and a cathode (an anode), respectively.

1. A heterocyclic compound of Formula 1: 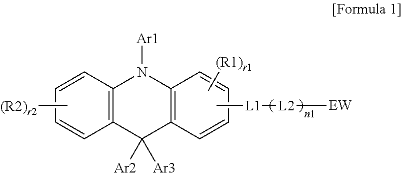
wherein:
L1 and L2 are the same as or different from each other, and are each independently a substituted or unsubstituted divalent heteroaryl group;
Ar1 to Ar3 are the same as or different from each other, and are each independently a substituted or unsubstituted alkyl group, a substituted or unsubstituted aryl group, or a substituted or unsubstituted heteroaryl group;
EW has a structure that serves as an electron acceptor;
R1 and R2 are the same as or different from each other, and are each independently hydrogen, deuterium, a halogen group, a nitrile group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylamine group, or a substituted or unsubstituted aryl group;
n1 is 0 or 1;
r1 is an integer from 1 to 3; and
r2 is an integer from 1 to 4.

2. The heterocyclic compound of claim 1, wherein Ar1 is a monocyclic or polycyclic aryl group.

3. The heterocyclic compound of claim 1, wherein Ar2 and Ar3 are the same as or different from each other, and are each independently a straight-chained or branched alkyl group.

4. The heterocyclic compound of claim 1, wherein L1 and L2 are the same as or different from each other, and are each independently selected from the following Formulae A to C: 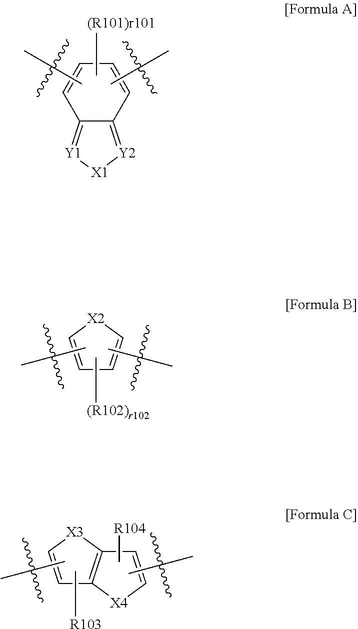
wherein:
X1 to X4 are the same as or different from each other, and are each independently O, S, or Se;
Y1 and Y2 are the same as or different from each other, and are each independently N or P;
R101 to R104 are the same as or different from each other, and are each independently hydrogen, deuterium, a halogen group, a nitrile group, a nitro group, a hydroxyl group, a carbonyl group, an ester group, an imide group, an amide group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group;
r101 and r102 are each 1 or 2;
when r101 is 2, multiple R101 are the same as or different from each other;
when r102 is 2, multiple R102 are the same as or different from each other; and 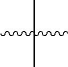
is a bond in Formula 1.

5. The heterocyclic compound of claim 1, wherein EW is selected from the following structures: 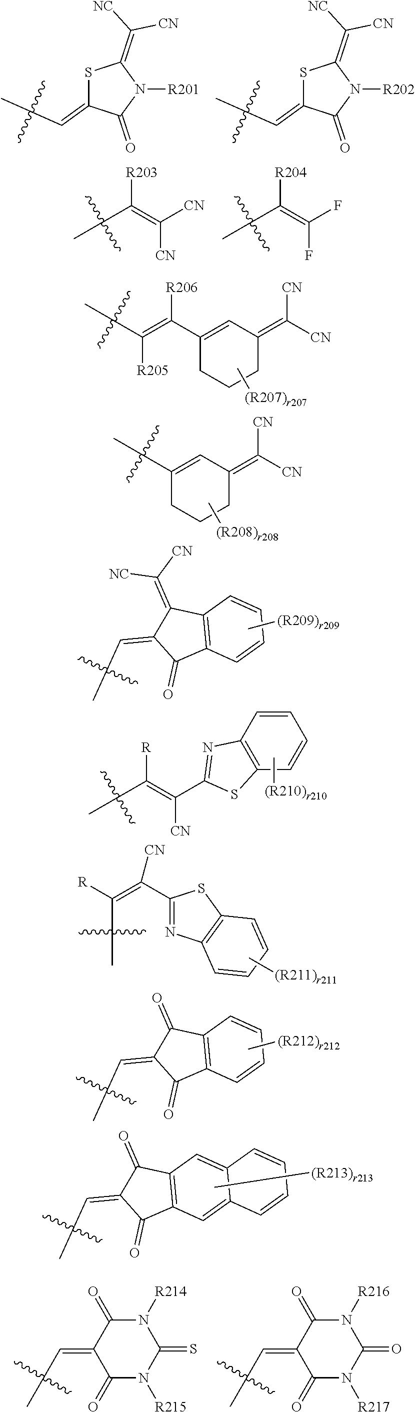 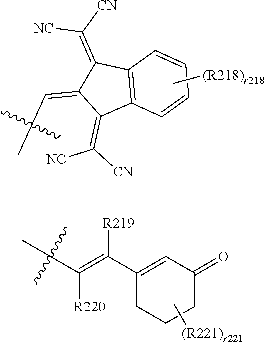
wherein:
R and R201 to R221 are the same as or different from each other, and are each independently hydrogen, deuterium, a halogen group, a nitrile group, a nitro group, a hydroxyl group, a carbonyl group, an ester group, an imide group, an amide group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group;
r207, r208, and r221 are each an integer from 1 to 7;
r209, r210, r211, r212, and r218 are each an integer from 1 to 4;
r213 is an integer from 1 to 6;
when r207 is 2 or more, multiple R207 are the same as or different from each other;
when r208 is 2 or more, multiple R208 are the same as or different from each other;
when r209 is 2 or more, multiple R209 are the same as or different from each other;
when r210 is 2 or more, multiple R210 are the same as or different from each other;
when r211 is 2 or more, multiple R211 are the same as or different from each other;
when r212 is 2 or more, multiple R212 are the same as or different from each other;
when r213 is 2 or more, multiple R213 are the same as or different from each other;
when r218 is 2 or more, multiple R218 are the same as or different from each other;
when r221 is 2 or more, multiple R221 are the same as or different from each other; and 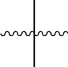
is a bond in Formula 1.

6. The heterocyclic compound of claim 1, wherein Formula 1 is a compound of any one of the following Formulae 1-1 to 1-4: 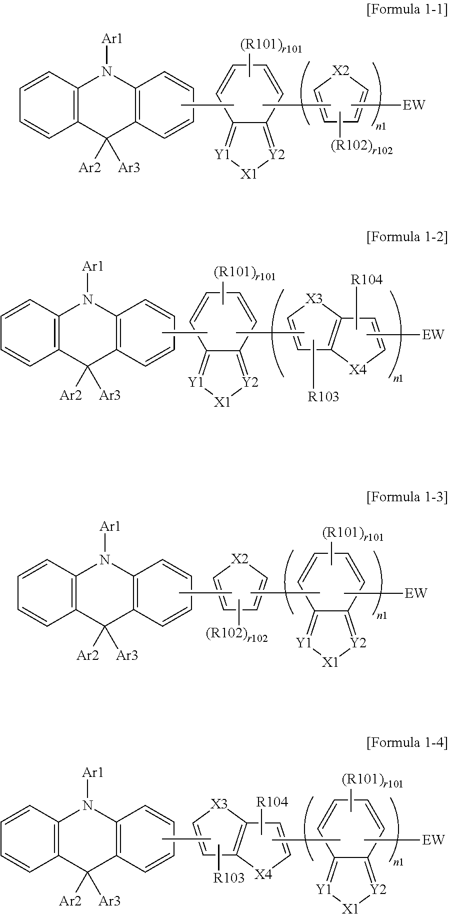
wherein:
the definitions of Ar1 to Ar3, n1, and EW are the same as those defined in Formula 1;
X1 to X4 are the same as or different from each other, and are each independently O, S, or Se;
Y1 and Y2 are the same as or different from each other, and are each independently N or P;
R101 to R104 are the same as or different from each other, and are each independently hydrogen, deuterium, a halogen group, a nitrile group, a nitro group, a hydroxyl group, a carbonyl group, an ester group, an imide group, an amide group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group;
r101 and r102 are each 1 or 2;
when r101 is 2, multiple R101 are the same as or different from each other; and
when r102 is 2, multiple R102 are the same as or different from each other.

7. The heterocyclic compound of claim 1, wherein Formula 1 is a compound of any one of the following Formulae 1-5 to 1-14: 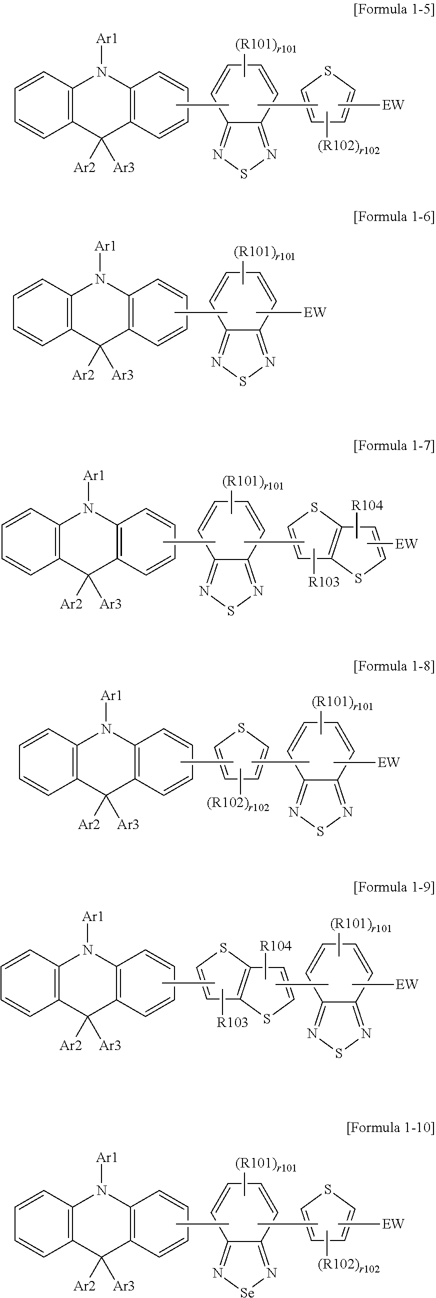 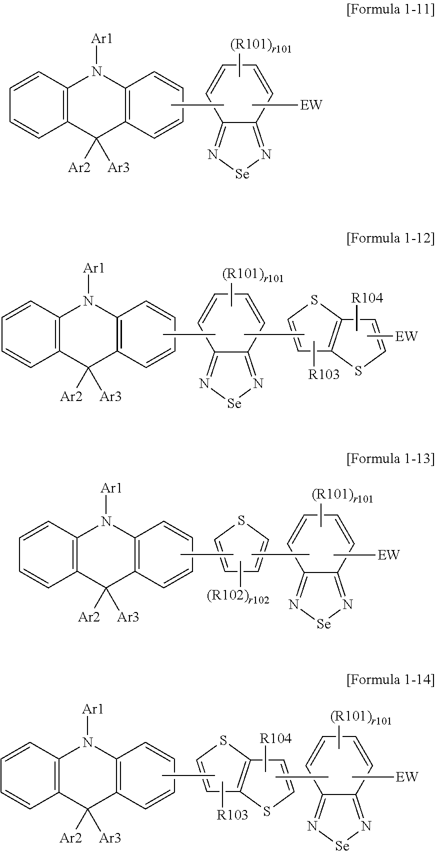
wherein:
the definitions of Ar1 to Ar3 and EW are the same as those defined in Formula 1;
R101 to R104 are the same as or different from each other, and are each independently hydrogen, deuterium, a halogen group, a nitrile group, a nitro group, a hydroxyl group, a carbonyl group, an ester group, an imide group, an amide group, a substituted or unsubstituted alkyl group, a substituted or unsubstituted cycloalkyl group, a substituted or unsubstituted alkoxy group, a substituted or unsubstituted aryloxy group, a substituted or unsubstituted alkylthioxy group, a substituted or unsubstituted arylthioxy group, a substituted or unsubstituted alkylsulfoxy group, a substituted or unsubstituted arylsulfoxy group, a substituted or unsubstituted alkenyl group, a substituted or unsubstituted silyl group, a substituted or unsubstituted boron group, a substituted or unsubstituted amine group, a substituted or unsubstituted arylphosphine group, a substituted or unsubstituted phosphine oxide group, a substituted or unsubstituted aryl group, or a substituted or unsubstituted heteroaryl group;
r101 and r102 are each 1 or 2;
when r101 is 2, multiple R101 are the same as or different from each other; and
when r102 is 2, multiple R102 are the same as or different from each other.

8. The heterocyclic compound of claim 1, wherein the compound of Formula 1 is selected from the following compounds: 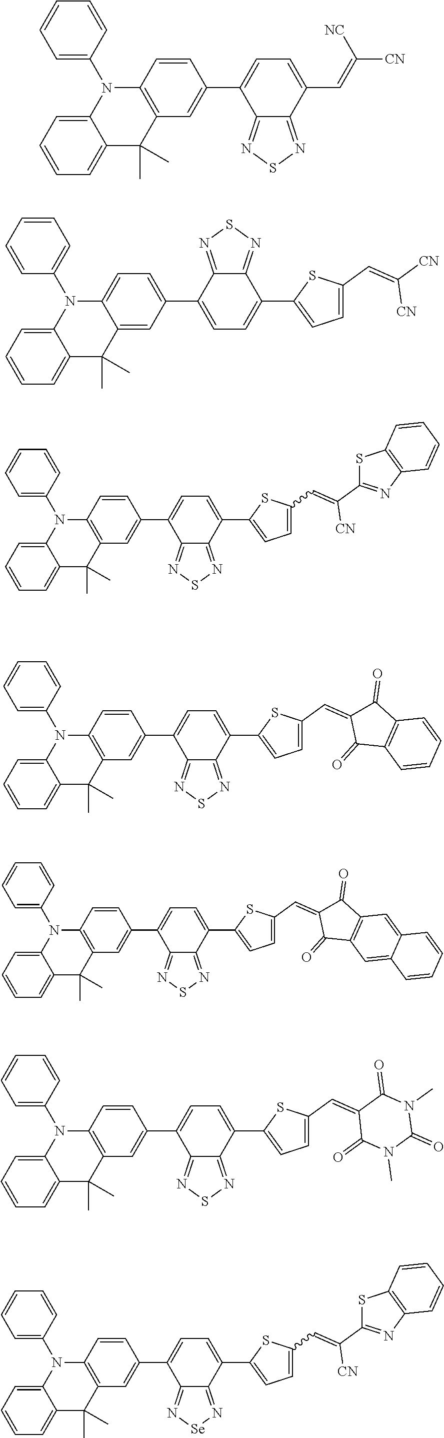 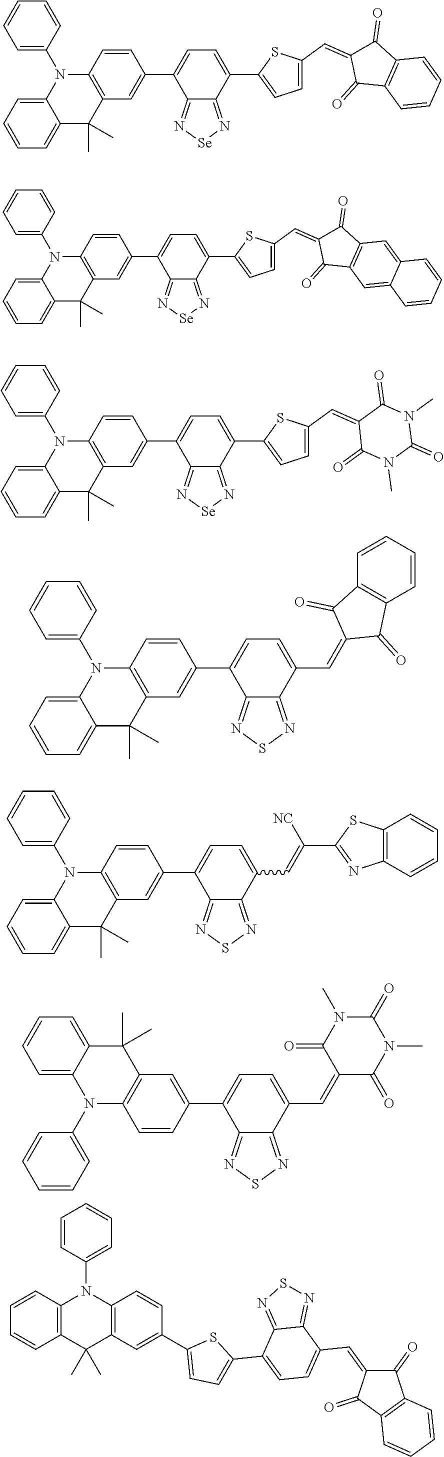 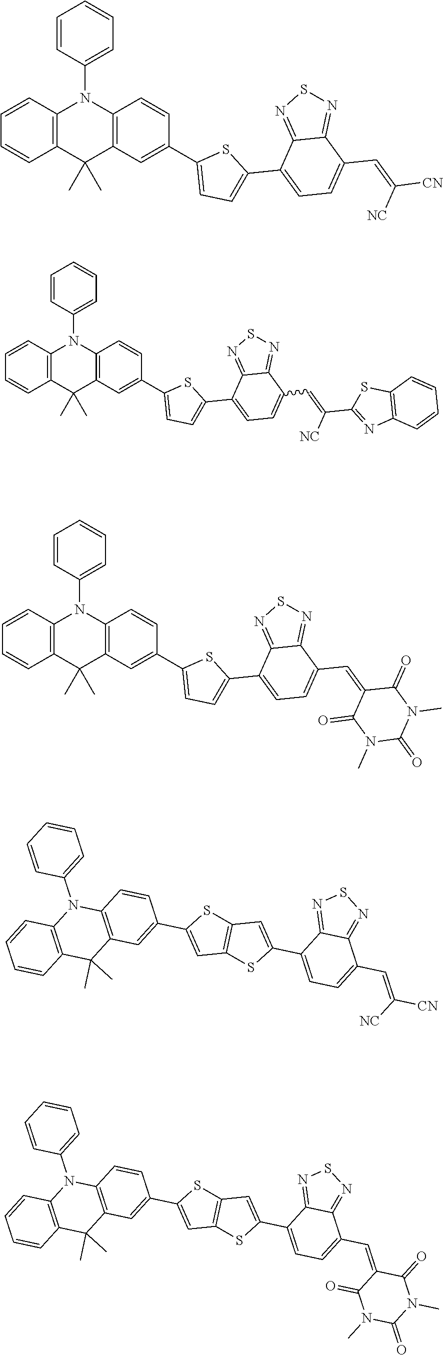 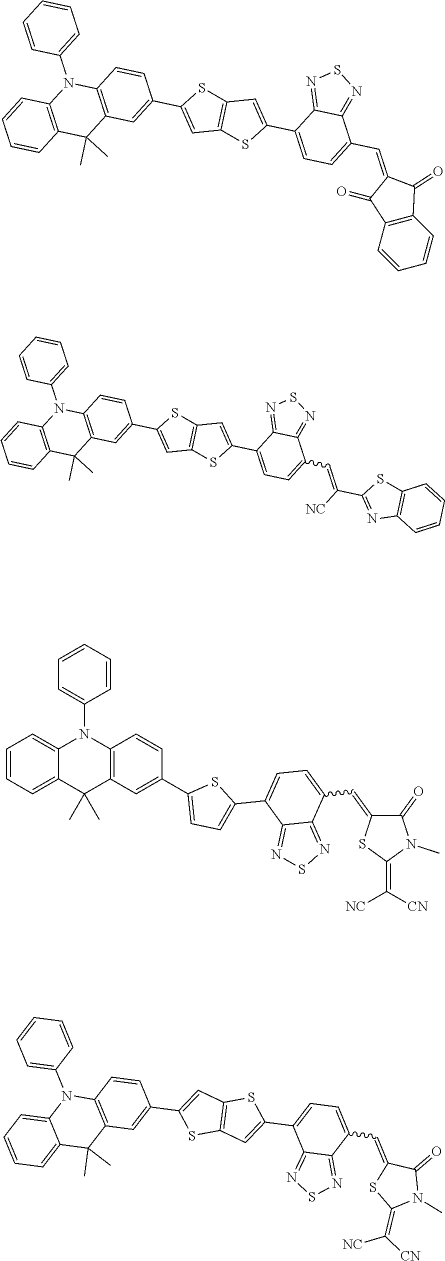 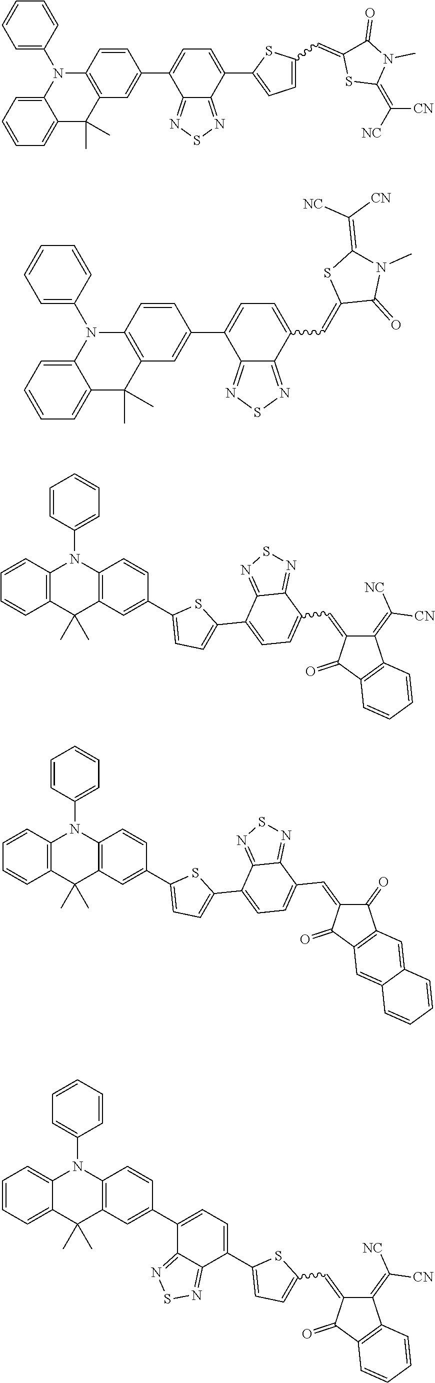 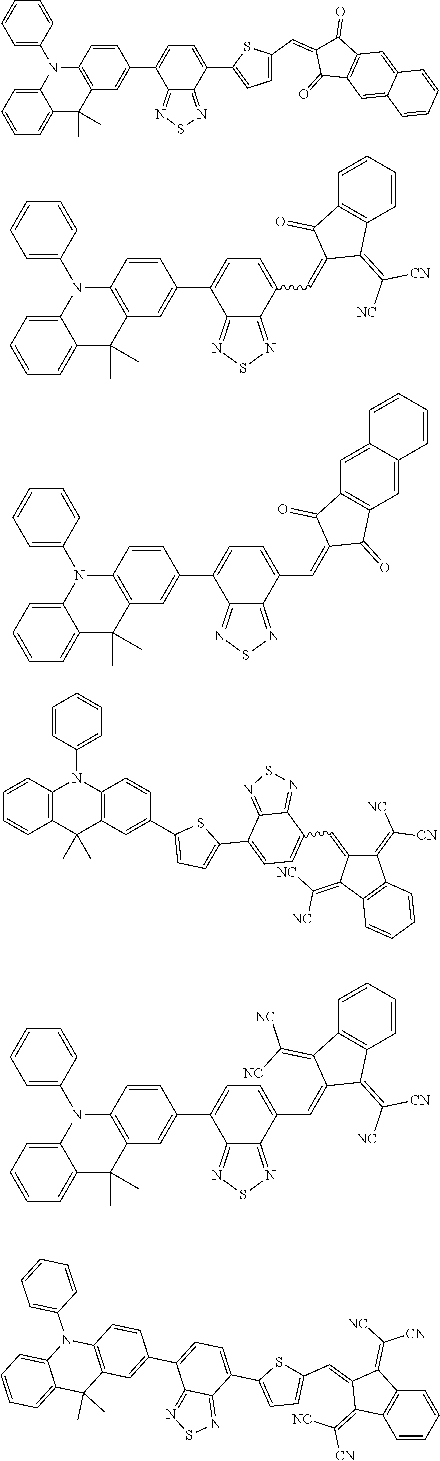 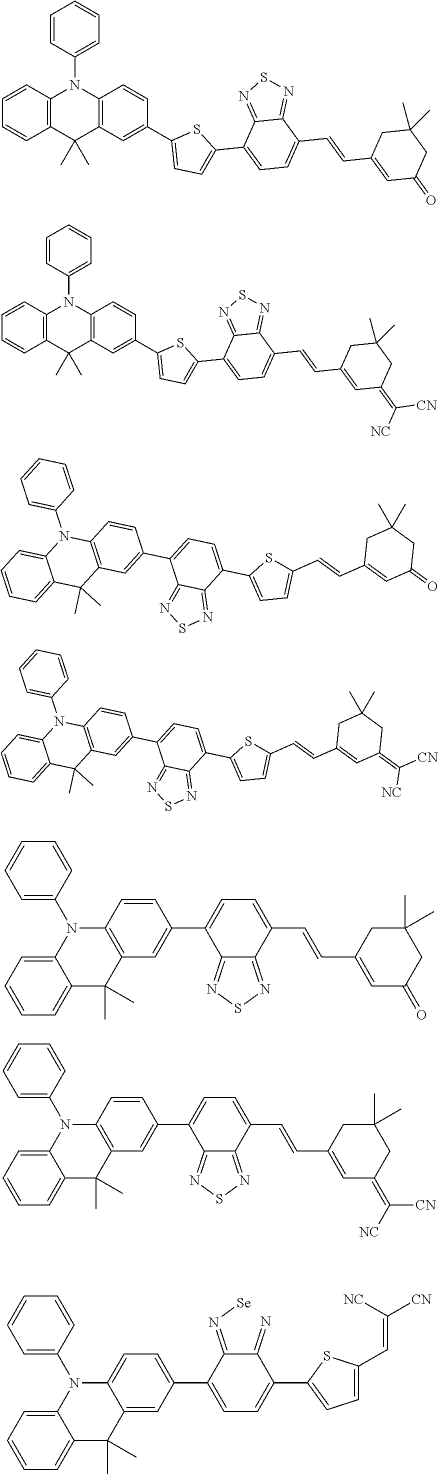
wherein [symbol] means that isomers having a trans structure and a cis structure are mixed with each other.

9. An organic photoelectric device comprising:
a first electrode;
a second electrode facing the first electrode; and
an organic material layer comprising one or more layers between the first electrode and the second electrode,
wherein the one or more layers of the organic material layer comprise the heterocyclic compound of claim 1.

10. The organic photoelectric device of claim 9, wherein the organic material layer comprises a photoactive layer,
the photoactive layer has a bilayer thin film structure comprising an n-type organic material layer and a p-type organic material layer, and
the p-type organic material layer comprises the heterocyclic compound.

11. The organic photoelectric device of claim 9, wherein the organic material layer comprises a photoactive layer,
the photoactive layer comprises an electron donor material and an electron acceptor material, and
the electron donor material comprises the heterocyclic compound.

12. The organic photoelectric device of claim 11, wherein the electron donor and the electron acceptor constitute a bulk heterojunction (BHJ).

13. An organic image sensor comprising the organic photoelectric device of claim 9.

14. An electronic device comprising the organic image sensor of claim 13.

15. The heterocyclic compound of claim 1, wherein:
r1 and r2 are each 1, and R1 and R2 are each hydrogen:
Ar1 is an unsubstituted aryl group; and
Ar2 and Ar3 are each an unsubstituted alkyl group.

16. The heterocyclic compound of claim 15, wherein Ar1 is a phenyl group.

17. The heterocyclic compound of claim 6, wherein:
Ar1 is an unsubstituted aryl group; and
Ar2 and Ar3 are each an unsubstituted alkyl group.

18. The heterocyclic compound of claim 17, wherein Ar1 is a phenyl group.

19. The heterocyclic compound of claim 7, wherein:
Ar1 is an unsubstituted aryl group; and
Ar2 and Ar3 are each an unsubstituted alkyl group.

20. The heterocyclic compound of claim 19, wherein Ar1 is a phenyl group.
